(19) United States
(12) Patent Application Publication
(10) Pub. No.: US 2007/0149868 A1
(43) Pub. Date: Jun. 28, 2007

(54) Method and Apparatus for Photostimulation Enhanced Analyte Property Estimation

(76) Inventors: Thomas B. Blank, Gilbert, AZ (US); Mutua Mattu, Chandler, AZ (US); Stephen L. Monfre, Gilbert, AZ (US); Marcy Markarewicz, Tempe, AZ (US)

Correspondence Address: GLENN PATENT GROUP, 3475 EDISON WAY, SUITE L, MENLO PARK, CA, 94025, US

(21) Appl. No.: 11/538,737

(22) Filed: Oct. 4, 2006

Related U.S. Application Data
(63) Continuation-in-part of application No. 10/841,200, filed on May 6, 2004.
(63) Continuation-in-part of application No. 10/472,856, filed on Sep. 18, 2003, now Pat. No. 7,133,710.
(60) Provisional application No. 60/362,885, filed on Mar. 8, 2002.
(60) Provisional application No. 60/724,060, filed on Oct. 5, 2005.

(30) Foreign Application Priority Data
Mar. 7, 2003 (US) PCT03/07065

(51) Int. Cl. A61B 5/00; A61B 6/00

(52) U.S. Cl. 600/316; 600/310; 600/473; 600/476

(57) ABSTRACT

A method and apparatus using photostimulation to treat or pretreat a sample site prior to analyte property estimation is presented. More particularly, photonic-stimulation at and/or near at least one sample site is used to enhance perfusion of the sample site leading to reduced errors associated with sampling. This allows an analyte property determination in well perfused regions of the body while sampling at a more convenient less well perfused region of the body.

### CROSS REFERENCE TO RELATED APPLICATIONS

This document:
is a continuation-in-part of U.S. patent application Ser. No. 10/472,856, filed Mar. 7, 2003, which claims benefit of PCT application no. PCT/US03/07065 filed Mar. 7, 2003, which claims benefit of U.S. provisional patent application No. 60/362,885 filed Mar. 8, 2002;
is a continuation-in-part of U.S. patent application Ser. No. 10/841,200 filed Mar. 7, 2003, which claims benefit of PCT application no. PCT/US03/07065 filed Mar. 7, 2003, which claims benefit of U.S. provisional patent application No. 60/362,885 filed Mar. 8, 2002; and
claims benefit of U.S. provisional patent application No. 60/724,060, filed Oct. 5, 2005;
each of which is incorporated herein in its entirety by this reference thereto.

### BACKGROUND OF THE INVENTION

1. Field of the Invention

The invention relates generally to biomedical methods and apparatus. More particularly, the invention relates to use of photonic-stimulation in combination with a blood/tissue analyte property estimation.

2. Discussion of the Prior Art

Blood is not uniformly distributed in the body. Even within the circulatory system, blood constituents are not uniformly distributed. For example, a blood borne species is added to blood and/or removed from blood as a function of position in the body. For example, oxygen is added in the lungs and removed at cells and glucose is present at different concentrations in poorly perfused regions as compared to well perfused regions due to differing rates of uptake and consumption of glucose at different locations in the body. Non-uniform distribution of blood borne analytes result in sampling errors or bias in biomedical calibrations and measurements.

### Glucose

Diabetes is a chronic disease that results in improper production and use of insulin, a hormone that facilitates glucose uptake into cells. While a precise cause of diabetes is unknown, genetic factors, environmental factors, and obesity appear to play roles. Diabetics have increased risk in three broad categories: cardiovascular heart disease, retinopathy, and neuropathy. Diabetics are predisposed to one or more of the following complications: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes: impaired glucose tolerance and hyperinsulinemia or hypoglycemia.

Diabetes Prevalence and Trends

Diabetes is an ever more common disease. The World Health Organization (WHO) estimates that diabetes currently afflicts 154 million people worldwide. There are 54 million people with diabetes living in developed countries. The WHO estimates that the number of people with diabetes will grow to 300 million by the year 2025. In the United States, 15.7 million people or 5.9 per cent of the population are estimated to have diabetes. Within the United States, the prevalence of adults diagnosed with diabetes increased by six percent in 1999 and rose by 33 percent between 1990 and 1998. This corresponds to approximately eight hundred thousand new cases every year in America. The estimated total cost to the United States economy alone exceeds $90 billion per year. Diabetes Statistics, National Institutes of Health, Publication No. 98-3926, Bethesda, Md. (November 1997).

Diabetes Management

Once diagnosed, long-term clinical studies have shown that the onset of diabetes related complications is significantly reduced through proper control of blood glucose concentrations. The Diabetes Control and Complications Trial Research Group, The effect of intensive treatment of diabetes on the development and progression of long-term complications in insulin-dependent diabetes mellitus, N Eng J of Med, 329:977-86 (1993). Long term control of glucose concentrations of non-insulin dependent diabetics has also been shown to reduce diabetes related complications. U.K. Prospective Diabetes Study (UKPDS) Group, Intensive blood-glucose control with sulphonylureas or insulin compared with conventional treatment and risk of complications in patients with type 2 diabetes, Lancet, 352:837-853 (1998); and Y. Ohkubo, H. Kishikawa, E. Araki, T. Miyata, S. Isami, S. Motoyoshi, Y. Kojima, N. Furuyoshi, M. Shichizi, Intensive insulin therapy prevents the progression of diabetic microvascular complications in Japanese patients with non-insulin-dependent diabetes mellitus: a randomized prospective 6-year study, Diabetes Res Clin Pract, 28:103-117 (1995). More recently, studies have indicated that testing and control of pre-diabetics leads to a significant delay of the onset of diabetes related complications.

### Glucose Concentration Measurement Types

Currently, blood glucose determination is categorized into four major types: traditional invasive, alternative invasive, noninvasive, and implantable. Due to the wide use of these modes of measurement and somewhat loose use of terminology in the literature, a summary of the terminology for each mode of measurement is provided herein in order to clarify usage within this document.

In the medical field, invasive often refers to surgery. That is not the definition of invasive herein. In the glucose concentration determination field, invasive is a term defined relative to noninvasive. Noninvasive refers to a method or use of an apparatus in which no biological sample or fluid is taken from the body in order to perform a glucose concentration measurement. Invasive then means that a biological sample is collected from the body. Invasive glucose concentration determinations are further broken into two separate groups. The first is a traditional invasive method in which a blood sample is collected from the body from an artery, vein, or capillary bed in the fingertips or toes. The second is an alternative invasive method in which a blood, interstitial fluid, or biological fluid sample is drawn from a region other than an artery, vein, or capillary bed in the fingertips or toes. A further description of these terms is provided in the remainder of this section.

1. Traditional Invasive Glucose Concentration Determination

There are three major categories of traditional or classic invasive glucose concentration determinations. The first two methodologies use blood drawn with a needle from an artery or vein, respectively. The third methodology uses capillary blood obtained via lancet from the fingertip, thumb, or toes. Over the past several decades, this has become the most common method for self-monitoring of blood glucose concentration at home, at work, or in public settings.

Typical glucose concentration analysis techniques include calorimetric and enzymatic glucose concentration analysis. The most common enzymatic based glucose concentration analyzers use glucose oxidase, which catalyzes the reaction of glucose with oxygen to form gluconolactone and hydrogen peroxide, equation 1. Glucose concentration determination uses techniques based upon depletion of oxygen in the sample, through the changes in sample pH, or via the formation of hydrogen peroxide. A number of calorimetric and electro enzymatic-techniques further use the reaction products as a starting reagent. For example, hydrogen peroxide reacts in the presence of platinum to form the hydrogen ion, oxygen, and current any of which are indirectly used to determine the glucose concentration, equation 2.
glucose+O₂→gluconolactone+H₂O₂ eq. 1
H₂O₂→2H⁺+O₂+2e⁻ eq. 2

To further clarify, an alternative invasive meter used to collect blood via lancet from sample sites consisting of the fingertip or toe is a traditional invasive glucose concentration analyzer.

2. Alternative Invasive Glucose Concentration Determination

There are several alternative invasive methods of determining glucose concentration.

A first group of alternative invasive glucose concentration analyzers have a number of similarities to the traditional invasive glucose concentration analyzers. One similarity is that blood samples are acquired with a lancet. This form of alternative invasive glucose concentration determination is not used to collect for analysis venous or arterial blood, but rather is used to collect capillary blood samples. A second similarity is that the blood sample is analyzed using chemical analyses that are similar to the calorimetric and enzymatic analyses describe above. The primary difference is that in an alternative invasive glucose concentration determination the blood sample is not collected from the fingertip or toes. For example, according to package labeling the TheraSense® FreeStyle Meter™ is preferably used to collect and analyze blood from the forearm. This is an alternative invasive glucose concentration determination due to the location of the lancet draw. In this first group of alternative invasive methods based upon blood draws with a lancet, a primary difference between the alternative invasive and traditional invasive glucose concentration determination is the location of blood acquisition from the body. Additional differences include: a gauge of a lancet; a depth of penetration of the lancet; timing issues; the volume of blood acquired; and environmental factors, such as the partial pressure of oxygen, altitude; and temperature. This form of alternative invasive glucose concentration determination includes any of: analysis of samples collected from the palmar region, base of thumb, forearm, upper arm, head, earlobe, torso, abdominal region, thigh, calf, and plantar region.

A second group of alternative invasive glucose concentration analyzers are distinguished by their mode of sample acquisition. This group of glucose concentration analyzers has a common characteristic of acquiring a biological sample from the body or modifying the surface of the skin to gather a sample without use of a lancet for subsequent analysis. For example, a laser poration based glucose concentration analyzer uses a burst or stream of photons to create a small hole in the surface of the skin. A sample of basically interstitial fluid collects in the resulting hole. Subsequent analysis of the sample for glucose concentration constitutes an alternative invasive glucose concentration analysis whether or not the sample was actually removed from the created hole. Herein, the term alternative invasive includes techniques that analyze biosamples physically removed from skin, such as interstitial fluid, whole blood, mixtures of interstitial fluid and whole blood, and selectively sampled interstitial fluid. An example of selectively sampled interstitial fluid is collected fluid in which large or less mobile constituents are not fully represented in the resulting sample. For this second group of alternative invasive glucose concentration analyzers sampling sites include: the hand, fingertips, palmar region, base of thumb, forearm, upper arm, head, earlobe, eye, chest, torso, abdominal region, thigh, calf, foot, plantar region, and toes. A number of methodologies exist for the collection of the sample for alternatively invasive measurements including laser poration, applied current, and suction. The most common are summarized here:
A. Laser poration: In these systems, photons of one or more wavelengths are applied to skin creating a small hole in the skin barrier. This allows small volumes of interstitial fluid to become available to a number of sampling techniques.
B. Applied current: In these systems, a small electrical current is applied to the skin allowing interstitial fluid to permeate through the skin.
C. Suction: In these systems, a partial vacuum is applied to a local area on the surface of the skin. Interstitial fluid permeates the skin and is collected.

In all of these techniques, the analyzed sample is interstitial fluid. However, some of the techniques are optionally applied to the skin in a fashion that draws blood. For example, the laser poration method alternatively results in blood droplets. In this document, any technique that draws biosamples from the skin without the use of a lancet on the fingertip or toes is referred to as alternative invasive technique.

Sometimes, the literature refers to an alternative invasive technique as an alternative site glucose concentration determination or as a minimally invasive technique. The minimally invasive nomenclature derives from the method by which the sample is collected. In this document, an alternative site glucose concentration determinations that draw blood or interstitial fluid, even a quarter microliter, are considered to be alternative invasive glucose concentration determination techniques as defined above. An example of an alternative invasive meter is the TheraSense® FreeStyle™ (Abbott, Abbott Park, Ill.) when not sampling fingertips or toes and equivalent technologies.

3. Noninvasive Glucose Concentration Determination

There exist a number of noninvasive approaches for glucose concentration estimation. These approaches vary widely, but have at least two common steps. First, an apparatus is used to acquire a reading from the body without obtaining a biological sample. Second, an algorithm is used to convert this reading into a glucose concentration estimation.

One species of noninvasive glucose analyzer is based upon spectra. Typically, a noninvasive apparatus uses some form of spectroscopy to acquire the signal or spectrum from the body.

4. Implantable Sensor for Glucose Concentration Determination

There exist a number of approaches for implanting a glucose concentration sensor into the body for glucose concentration determination. These implantables are used to collect a sample for further analysis or are used to acquire a reading of the sample directly or indirectly. For example, a wick placed subcutaneously to collect a sample overnight that is removed and analyzed for glucose content representative of the interstitial fluid glucose concentration is referred to as an implantable. Similarly, a biosensor or electrode placed under the skin is referred to as an implantable device.

An implantable analyzer reads from one or more of a variety of body fluids or tissues including but not limited to: arterial blood, venous blood, capillary blood, interstitial fluid, and selectively sampled interstitial fluid. For example, an implantable glucose concentration analyzer is placed transcutaneously; in the peritoneal cavity; in an artery; in muscle; or in an organ, such as the liver or brain. The implantable glucose sensor is one component of an artificial pancreas.

### Glucose Distribution

A number of reports are summarized herein that indicate that the glucose concentration in alternative sites, such as the forearm, differ from those of traditional sample sites, such as the fingertip. Additional reports indicate that alternative site glucose concentrations are equivalent to fingerstick glucose concentration determination.

Szuts from Abbott Laboratories concluded that measurable physiological differences in glucose concentration between the arm and fingertip exist, but that these differences were found to be clinically insignificant even in those subjects in whom they were measured. [Szuts, Ete Z.; Lock, J. Paul; Malomo, Kenneth J.; Anagnostopoulos, Althea “Blood Glucose Concentrations of Arm and Finger During Dynamic Glucose Conditions”, Diabetes Technology & Therapeutics, 4, 2002, 3-11].

Lee, from Roche Diagnostics Corporation, concluded that patients testing two-hours postprandial are expected to see small differences between their forearm and fingertip glucose concentrations. [Lee, Debra M.; Weinert, Sandra E.; Miller, Earl E. “A Study of Forearm Versus Finger Stick Glucose Monitoring”, Diabetes Technology & Therapeutics, 4, 2002, 13-23].

McGarraugh from TheraSense, Inc. concluded that there is no significant difference in HbA₁C measurements for patients using alternative site meters on the fingertip and traditional glucose concentration analyzers used on the fingertip. [Bennion, Nancy; Christensen, Nedra K.; McGarraugh, Geoff “Alternate Site Glucose Testing: A Crossover Design”, Diabetes Technology & Therapeutics, 4, 2002, 25-33].

Peled from Amira Medical concluded that glucose concentration monitoring of blood samples from the forearm is suitable when expecting steady state glycemic conditions and that the palm samples produced a close correlation with fingertip glucose concentration determinations under all glycemic states. [Peled, Nina; Wong, Daniel; Gwalani, Shilpa “Comparison of Glucose Levels in Capillary Blood Samples from a Variety of Body Sites”, Diabetes Technology & Therapeutics, 4, 2002, 35-44].

Based upon a study using fast acting insulin injected intravenously, Koschinsky suggested that to avoid risky delays of hyperglycemia and hypoglycemia detection that monitoring at the arm is preferably limited to situations in which ongoing rapid changes in the blood glucose concentration are excluded. [Jungheim, Karsten; Koschinsky, Theodor “Glucose Monitoring at the Arm”, Diabetes Care, 25, 2002, 956-960 and Jungheim, Karsten; Koschinsky, Theodor “Risky Delay of Hypoglycemia Detection by Glucose Monitoring at the Arm”, Diabetes Care, 24, 2001, 1303-1304].

Equilibration Approaches

While there exist multiple reports that glucose concentrations are very similar when collected from the fingertip or alternative locations, a number of sampling approaches have been recommended to increase localized perfusion at the sample site to equilibrate the values just prior to sampling. Several of these approaches are summarized here:
1. Pressure: One sampling methodology requires rubbing or applying pressure to the sampling site in order to increase localized perfusion prior to obtaining a sample via lancet. An example of this is TheraSense's FreeStyle blood glucose concentration analyzer. [McGarraugh, Geoff; Schwartz, Sherwyn; Weinstein, Richard “Glucose Measurements Using Blood Extracted from the Forearm and the Finger”, TheraSense, Inc., ART01022 Rev. C, 2001 and McGarraugh, Geoff; Price, David; Schwartz, Sherwyn; Weinstein, Richard “Physiological Influences on Off-Finger Glucose Testing”, Diabetes Technology & Therapeutics, 3, 2001, 367-376].
2. Heating: Heat applied to the localized sample site has been proposed as a mechanism for equalizing the concentration between the vascular system and skin tissue by dilating the capillaries allowing more blood flow, which leads toward equalization of the venous and capillary glucose concentrations. Alternatively, vasodilating agents, such as nicotinic acid, methyl nicotinamide, minoxidil, nitroglycerin, histamine, capsaicin, or menthol is used to increase local blood flow. [Rohrscheib, Mark; Gardner, Craig; Robinson, Mark R. “Method and Apparatus for Noninvasive Blood Analyte Measurement with Fluid Compartment Equilibration”, U.S. Pat. No. 6,240,306, May 29, 2001].
3. Vacuum: Applying a partial vacuum to the skin at and around the sampling site prior to sample collection has also been used. A localized deformation in the skin allows superficial capillaries to fill more completely. [Ryan, T. J. “A Study of the Epidermal Capillary Unit in Psoriasis”, Dermatologica, 1969, 138, 459-472] For example, Abbott uses a vacuum device at one-half atmosphere that pulls the skin up 3.5 mm in their integrated device. Abbott maintains this deformation results in increased perfusion that equalizes the glucose concentration between the alternative site and the fingertip. [Ng, Ron Presentation to the FDA at the Clinical Chemistry & Clinical Toxicology Devices Panel Meeting, Gaithersburg, Md. Oct. 29, 2001].
4. Vasodilating Agent: The application of topical pharmacologic or vasodilating agents, such as nicotinic acid, methyl nicotinamide, minoxidil, nitroglycerin, histamine, menthol, capsaicin, and mixtures thereof is described as hastening the equilibration of the glucose concentration in the blood vessels with that of the interstitial fluid [see Rohrscheib, Mark; Gardner, Craig; Robinson, Mark R. Method and Apparatus for Non-invasive blood analyte measurement with Fluid Compartment Equilibration, U.S. Pat. No. 6,240,306, May 29, 2001 and Robinson, Mark Ries; Messerschmidt, Robert G. Method for Non-Invasive Blood Analyte Measurement with Improved Optical Interface, U.S. Pat. No. 6,152,876, Nov. 28, 2000].

### Noninvasive Glucose Concentration Estimation

There exist a number of reports on noninvasive glucose concentration analysis technologies. Some of these relate to general instrumentation configurations required for noninvasive glucose concentration determination. Others refer to sampling technologies. Those related to the present invention are briefly reviewed here:

Barnes, U.S. Pat. No. 5,379,764 describes the advantages more frequent analysis using noninvasive analyzers resulting in tighter control of blood glucose concentrations.

General Instrumentation

Robinson, U.S. Pat. No. 4,975,581 describes a method and apparatus for measuring a concentration of a biological analyte, such as glucose, using infrared spectroscopy in conjunction with a multivariate model. The method is a two-step method that having both a calibration and a prediction step.

Malin, U.S. Pat. No. 6,040,578 describes a method and apparatus for determination of an organic blood analyte using multi-spectral analysis in the near-infrared. A plurality of incident wavelengths are incident upon a sample surface, diffusely reflected radiation is collected, and the analyte concentration is determined via chemometric techniques.

Complications Related to Non-uniform Blood Analyte Distribution

The body is dynamic in nature. Body constituents are subject to input and output events that occur at non-uniform times and in fashions that are not equally distributed through the body. This results in certain body constituents constantly being in a state of flux. For example, the glucose concentration in the body is not equally distributed in different body compartments or within the circulatory system.

Non-uniform blood analyte distribution within the circulatory system leads to several problems. First, the state of the body often hinders noninvasive measurements. For example, localized circulation and temperature often negatively impact the state of the skin. A change of skin state can severely impact an optical reading of skin resulting in decreased precision and/or accuracy of a noninvasive analyzer, such as oxygen saturation determinations or glucose concentration estimations. What is needed is a mechanism to enhance localized circulation. Second, difficulties arise when one portion of the body is sampled to determine or measure a constituent concentration when it is desirable to determine the concentration of that constituent in a different body part. For example, glucose concentration is measured at an alternative site, such as the forearm, when it is desirable to determine the fingertip, arterial, or venous glucose concentration. Medical treatment and diagnosis protocols are often developed using reference analyte concentrations collected from well perfused body parts, such as from arterial, venous, or fingertip blood. This leads to complications when subsequent tests collect blood or analyze blood/tissue at different locations within the body. As a result of non-uniform distribution of a blood analyte, such as glucose within the circulatory system, error is introduced into a resultant analysis if standard medical diagnosis and subsequent treatment protocols are utilized. What is needed is a method allowing use of standard diagnostic and treatment protocols using data collected from alternative sites or using alternative methods.

An example is illustrative of the problem. Within the body, site-to-site variation in glucose concentration within the circulatory system results in problems when a medical diagnosis and subsequent treatment relies on the concentration of the analyte to be the same at within different body parts. For instance, standard medical treatments for high and low glucose concentrations are to administer insulin or intake glucose, respectively. However, the medical procedure is based upon blood samples taken from a well perfused region of the body. If the blood glucose concentration is taken from another region of the body, then differences in blood glucose concentration between a traditional site and an alternative site can lead to instances where the standard or calibration does not wholely apply and resultant treatments are in error. For example, the alternative site glucose concentration is often higher than arterial blood glucose concentration when the glucose concentration in the well perfused regions of the body is dropping rapidly. As a result, a reading of glucose concentration from an alternative site will be artificially high and the medical treatment of administering insulin can further reduce a subject's glucose concentration to dangerously low or even fatal concentrations. What is needed is the ability to reduce site-to-site variation in blood borne constituents in order to reduce or eliminate sampling or measurement problem associated with difference in site-to-site variation in the constituent. The invention provides a method and apparatus for enhancing perfusion of capillary, tissue, or skin layers to optimize the tissue for noninvasive analysis.

### SUMMARY OF THE INVENTION

A method and apparatus using photostimulation to treat or pretreat a sample site prior to analyte property estimation is presented. More particularly, photonic-stimulation at and/or near at least one sample site is used to enhance perfusion of the sample site leading to reduced errors associated with sampling. This allows an analyte property determination in well perfused regions of the body while sampling at a more convenient less well perfused region of the body.

### BRIEF DESCRIPTION OF THE DRAWINGS

FIG. 1 is a graph that shows a dampening and lag in forearm glucose concentration profile versus a fingertip reference profile according to the invention;

FIG. 2 is a graph that shows improved correlation in glucose concentration profiles between a photo-stimulated site and a reference fingertip compared to a non-photo-stimulated site according to the invention;

FIG. 3 is a graph that shows that noninvasive glucose concentration determinations performed at photo-stimulated sites predict with increased accuracy the capillary blood glucose concentration versus alternative site blood glucose concentration according to the invention;

FIG. 4 is a graph that shows noninvasive glucose concentration predictions from photo-stimulated sites versus capillary glucose reference concentrations according to the invention;

FIG. 5 is a graph that shows predictions from an untreated site using a noninvasive glucose concentration analyzer versus a fingertip reference for six subjects;

FIG. 6 is a graph that shows predictions from a treated site using a noninvasive glucose concentration analyzer versus a fingertip reference for six subjects according to the invention;

FIGS. 7a-7d provide perspective, schematic views of an LED plug attachment coupled to a guide according to the invention;

FIGS. 8a and 8b present a schematic representation of sample probe control and sample probe movement relative to a sample;

FIG. 9 illustrates a block diagram of an analyzer having two primary systems, a targeting system and a measuring system;

FIG. 10 illustrates a proximity sensor having a plurality of detection points for estimating distance to contact with a sample site and/or contact with the sample site;

FIG. 11A and FIG. 11B present a sample probe having a tilt adjustable mechanism in two states; and

FIG. 12A and FIG. 12B present a sample probe in two tilt configurations, respectively.

### DETAILED DESCRIPTION OF THE INVENTION

The invention comprises the use of photostimulation in conjunction with an analyte property estimation of a blood or tissue constituent. More particularly, photostimulation at or near at least one sample site is used to enhance perfusion of a sample site. Increased perfusion at the sample site decreases site-to-site variation in the blood analyte property within the body. Hence, sampling at sample sites not used to generate a calibration, methodology, or protocol results in fewer sampling errors or a smaller magnitude of sampling error. This results in an increased accuracy of the calibration, methodology, or protocol in measurement and/or use of the estimated or determined sample constituent property.

In a preferred embodiment of the invention photostimulation of a sample site is used to minimize site-to-site analyte property differences within a body in conjunction with an invasive, minimally invasive, or noninvasive blood analyte property estimation or determination.

In another embodiment of the invention, photostimulation is used to reduce site-to-site analyte property differences within a body to minimize sampling errors associated with a calibration or treatment methodology.

In still another embodiment of the invention, photostimulation is used to increase an analyte bearing tissue volume for subsequent or concurrent noninvasive sampling and analysis.

In yet additional embodiments of the invention, photostimulation is used in combination with means for enhancing tissue perfusion, such as intake of L-arginine, temperature control, application of a partial vacuum, addition of a vasodilating agent, and/or application of pressure or rubbing at or about the sampled tissue site. Wile glucose concentration is used herein as an illustrative example, the invention relates to the use of photonic-stimulation to enhance perfusion prior to or during analyte determination of any blood and/or tissue constituent.

Many examples of photostimulation in combination with noninvasive analyses are presented herein and are described in more detail, infra. Photostimulation is used to treat a tissue sample site prior to or during an analysis of the tissue site. In the case of noninvasive oxygen saturation estimation, photostimulation increases localized blood perfusion. The increased blood perfusion enhances the sample volume containing the analyte that is probed by the pulse oximeter, increases the ability to determine pulsatile flow, stabilizes temperature, and increases the localized hemoglobin concentration. In the case of noninvasive glucose concentration estimation, in addition to the above described effects relating to perfusion, glucose concentration at an alternative site is adjusted toward the glucose concentration at a well perfused sample site. In general, the increased sample site perfusion is used to enhance the state of the sample for noninvasive sampling. Additional sample constituents of interest include but are not limited to: fats, such as triglycerides or forms of cholesterol; proteins, such as albumin or globulin; urea; bilirubin; hemoglobin; deoxyhemoglobin; oxygen; electrolytes, such as Na⁺, Ca²⁺, and K⁺; and chelates. Additional means for stimulating localized perfusion are optionally used in conjunction with photostimulation. Examples of photostimulation in conjunction with noninvasive oxygen saturation estimation, noninvasive glucose concentration estimation, and angiogenesis are used to illustrate the invention, but are not intended to limit the scope of the invention.

### Photostimulation

Nitric Oxide

Nitric oxide (NO) is used to induce vasodilation. Nitric oxide is a free radical gas that behaves as an endogenous vasodilator, which is important in regulation of circulation. Nitric oxide initiates and maintains vasodilation through a cascade of biological events that culminate in the relaxation of smooth muscle cells that line arteries, veins, and lymphatics [see Furchgott, R. F. Nitric Oxide: From Basic Research on Isolated Blood Vessels to Clinical Relevance in Diabetes”, An R Acad Nac Med (Madrid), 1998, 115, 317-331]. The sequence of biological events that are triggered by NO includes:
Step 1: NO gas released from nitrosothiols in hemoglobin or from endothelial cells, diffuses into smooth muscle cells that line small blood vessels;
Step 2. once inside the smooth muscle cell, NO binds to an enzyme, called guanylate cyclase (GC) and this binding results in GC activation;
Step 3. activated GC cleaves two phosphate groups from another compound called guanosine triphosphate (GTP) resulting in the formation of cyclic guanosine monophosphate (cGMP) that is used to phosphorylate proteins, including the smooth muscle contractile protein called myosin; and
Step 4. once phosphorylated, the smooth muscle cell myosin relaxes, resulting in dilation of the vessel that was originally exposed to NO.

Essentially, nitric oxide is a signaling molecule, which is known to relax smooth muscle in arteries, veins, and lymph vessels. When these vessel muscles relax they dilate, which results in increased circulation through decreased resistance [see Carnegie, Dale “The Use of Monochromatic Infrared Energy Therapy in Podiatry, Podiatry Management”, November/December 2002, 129-134].

Photostimulation

Photostimulation is also referred to as photo-stimulation, photonic-stimulation, or stimulation or excitation with light or photons. Photostimulation is herein used as photons being absorbed by an absorber that subsequently releases an agent that results in increased perfusion. Photostimulation is distinct from photonic heating. Photonic heating is optionally used in conjunction with photostimulation.

Photostimulation at or near the sample site is performed in a manner that enhances perfusion of the sample site primarily by enhancing or inducing perfusion of the sample site. Nitric oxide is stored in cells, such as red blood cells. The release of nitric oxide when white light is presented to tissues results in increased blood flow. Because light is made up of several different wavelengths, research studies explored the beneficial effects of individual wavelength to determine which might be better at causing NO production or release thus stimulating vasodilation. Studies with visible colors were followed by experiments with monochromatic sources of non-visible light, such as ultraviolet and near-infrared light.

Generally, photostimulation devices use near-infrared light at about 890 or 910 nm to accomplish the local release of NO from hemoglobin and possibly other heme proteins within red blood cells. Carnegie, supra, describes the use of monochromatic light at about 890 nm to stimulate NO release. Noble, Gareth J.; Lowe, Andrea S.; Baxter, David G. Monochromatic Infrared Irradiation (890 nm): Effect of a Multisource Array upon Conduction in the Human Median Nerve, J. of Clin. Laser Medicine and Surgery, 2001, 19, 291-295 also describe use of 890 nm light to induce stimulation. Examples of photostimulation devices are those produced by Anodyne Therapy, LLC (Tampa, Fla.).

Release of nitric oxide via photostimulation is also used for pain mediation, wound healing, tissue repair, and to increase circulation with implications to medical treatment of circulatory related problems associated with ulcers, eyes, kidneys, the heart, and the intestine.

Photostimulation is used to pretreat or concurrently treat a tissue sample site in combination with noninvasive analyte property estimation. Photostimulation results in any of:
enhanced localized perfusion;
increase optical pathlength of analyte containing tissue;
enhanced equilibration of an analyte in poorly perfused and well perfused regions; and
enhanced pulsatile flow.

The changes in the localized state of the sampled tissue site due to photostimulation result in increased optical signal allowing enhanced precision and/or accuracy of an estimated analyte property value, such as oxygen saturation or glucose concentration.

### Noninvasive Analyte Property Estimation

Parameters affecting noninvasive analyte property estimation are described here.

Analyte Distribution

Constituents of blood and/or tissue that are acquired from outside sources, generated, or consumed are not equally distributed in the body. For example, it is well known that the oxygen concentration of arterial blood is greater after the lungs compared with the venous blood returning to the lungs. Still lower oxygen concentrations are found in poorly perfused regions of the body. Generally, analytes that are picked up or dropped off by blood have different concentrations in different portions of the body at the same time due to the localized rate of change of the constituent being faster than the replenishing or equalizing circulatory flow at less well perfused sites. For example, the concentration of a blood constituent near the skin surface often differs from that of the concentration of the same analyte in the well perfused regions of the circulatory system. Concentrations of analytes in interstitial fluid are also dependent upon the perfusion of nearby regions. For example, the concentration of glucose in interstitial fluid decrease as a function of time due to glycolysis. The decrease in glucose concentration is dependent upon both distance from a capillary bed and the history of perfusion of the capillary bed. Hence, as the perfusion of nearby regions is enhanced, it affects both capillary and interstitial glucose concentrations. Often, it is desirable to measure or determine the general concentration of such an analyte in the body while sampling at a localized site is preferred.

Oxygen Distribution

Oxygen is transported in the body by bonding and releasing from hemoglobin transported by the blood. Only two to three percent of all oxygen carried in the blood is dissolved in plasma. As discussed, supra, the oxygen saturation in blood is largest after exiting the lungs and decreases as oxygen is delivered to body components. The rate of blood flow is a factor in the rate of delivery. If the localized perfusion is diminished, then the hemoglobin delivers, relative to the flow rate, more oxygen locally for consumption and the localized oxygen saturation as measured by the oxy/deoxyhemoglobin ratio decreases. Hence, in a poorly perfused region, the localized estimation of oxygen saturation using a pulse oximeter is lower than the oxygen saturation at many well perfused regions. Accordingly, the localized oxygen saturation is not representative of the generalized oxygen saturation of the body.

A number of parameters lead to poor localized perfusion, such as hypotension, vasoconstriction, and hypothermia, which all lead to reduced localized levels of oxygen saturation. This leads to inaccurate total body readings of oxygen saturation. In addition, parameters leading to poor localized perfusion lead to reduced pulsatility of capillary blood often used in pulse oximetry. This leads to inaccurate readings of oxygen saturation or no reading at all due to the reliance of many pulse oximeters on determination of pulse in their algorithms for isolating the oxygen saturation percentage.

As discussed, infra, photostimulation is used to enhance localized perfusion to minimize poor circulation effects. As a result of the increased perfusion due to photostimulation, pulse oximetry readings have increased precision and/or accuracy in a region treated with photostimulation. From a signal-to-noise ratio perspective, increased perfusion at the sample site results in a higher percentage of the sampled volume being blood, which correlates to a larger optically probed pathlength having hemoglobin, which is a molecule yielding an analytical signal of interest. Therefore, photostimulation increases the pathlength of analyte containing tissue. Under the simplified analysis of Beer's Law, this results in a larger signal and thus enhances precision and accuracy of noninvasive oxygen saturation determination. Similarly, the increased percentage of blood in a sample volume results in a larger optical signal for all blood borne analytes when a noninvasive probe optically samples the region having enhanced perfusion.

The inventors have further recognized that photostimulation induced perfusion enhances the hydration of the stratum corneum as a result of enhanced influx of water into the well perfused tissue volume that acts against the dehydration of the skin surface from evaporation, which results in a highly order surface of keratinocytes. For optical based noninvasive analyzers, this allows for higher light throughput into the sample as a result of decreased surface scatter of incident photons. In addition, the ordered surface also reduces sample site variability as a function of time, such as through a measurement day.

Glucose Distribution

Glucose is concentrated in aqueous based body compartments. Further, within aqueous body compartments, glucose is not evenly distributed. Certainly intracellular and extracellular glucose concentrations differ. In addition, intra-vascular glucose concentration is often different in various parts of the body at the same time. Generally, the circulatory system moves blood glucose rapidly through the main arterial/venous channels. In well perfused capillary beds, such as the fingertips, the glucose concentration is roughly equivalent to that of the main arterial and venous compartments. Generally speaking, the concentration of glucose is uniform in the main arterial/venous circulatory system, though some glucose is consumed by the body, such that in some cases arterial glucose concentration exceeds that of venous glucose concentration. Again, some glucose is used in the capillary regions that decreases the localized glucose concentration. However, as the perfusion rates are large the glucose concentrations do not vary considerably.

Some regions of the body are not as well perfused as that of the fingertip. Generally speaking, less well circulated or perfused regions are more likely to have periods in which the glucose concentration differs from the more well perfused regions of the body. Differences result from glucose metabolism or synthesis. Again, it is often desirable to measure or determine the general concentration of glucose in the body with a test at a localized site. One method of increasing the localized perfusion so as to obtain a more representative sample is prior or concurrent photostimulation of the tissue sample site, as described herein.

A detailed description of glucose concentration differences between traditional invasive sites, such as the fingertip and alternative invasive sites, such as the forearm has been previously provided in U.S. application Ser. No. 10/377,916, which is incorporated herein in its entirety by this reference thereto.

It has been determined that differences between traditional invasive and alternative invasive glucose concentration determinations exist. It is further determined that the differences between the alternative invasive glucose concentration from a site, such as the forearm and the glucose concentration from a traditional invasive fingerstick vary as a function of at least time and location. Several example illustrate this point.

### EXAMPLE I

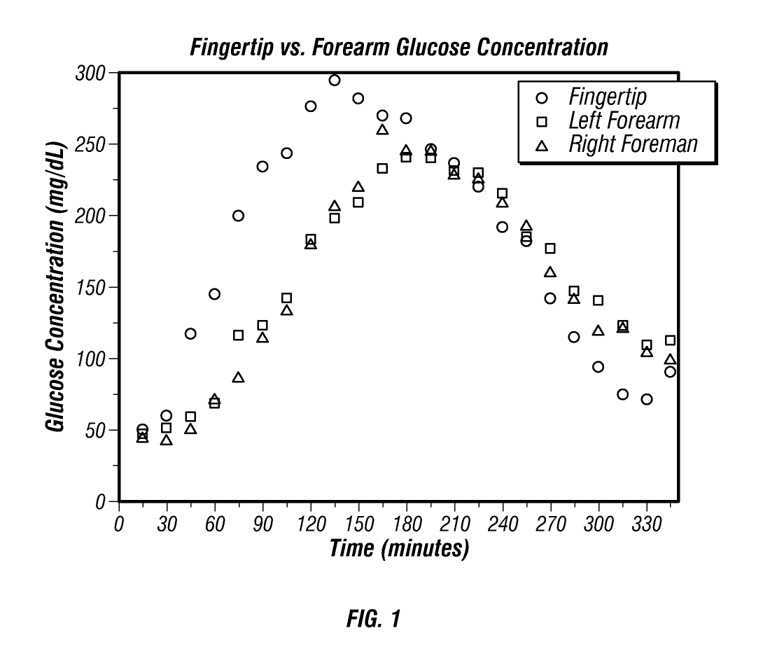

Referring now to FIG. 1, variations of glucose concentration at locations in the body is demonstrated. A diabetic subject was run through a glucose concentration perturbation. Over a period of four hours the glucose concentration started low at around 80 mg/dL, was increased to circa 350 mg/dL, and was brought back to circa 80 mg/dL. This profile was generated with intake of approximately seventy-five grams of a liquid form of carbohydrate in combination with subsequent injection of insulin to generate an ‘n’ glucose concentration profile. Traditional invasive fingertip capillary glucose concentrations were determined every fifteen minutes through the four-hour protocol and were followed quickly in time with alternative invasive capillary glucose concentration determinations with samples collected from the volar aspect of the subject's right and then left forearm. This resulted in 69 data points. The resulting glucose concentration profiles are presented in FIG. 1. The alternative invasive glucose concentrations measured at the forearm are demonstrated to be substantially dampened in magnitude and have a lagged profile in terms of initial rise, peak intensity position, and subsequent fall compared to the corresponding fingertip glucose concentration profile.

Several conclusions are drawn from this and previously presented data. First, during a glucose concentration excursion, substantial differences are sometimes observed between the capillary blood glucose concentration of the untreated forearm and the fingertip. Second, rapid changes in blood glucose concentration magnify differences between the measured blood glucose concentration of the fingertip and forearm while the relative errors are proportional to the glucose concentration. Third, during periods of rapid change in blood glucose concentration, differences between the forearm and fingertip give rise to a higher percentage of points in less desirable regions of a Clarke error grid. Fourth, the measured blood glucose concentrations of the volar aspect of the left and right forearms are similar. Finally, these findings are consistent with the mechanism of decreased perfusion into the forearm versus that of the fingertip leading to a dampening and/or lag in the glucose concentration profile versus well perfused regions.

Physiology

The above described conclusions on oxygen and glucose concentration distribution are consistent with the circulatory physiology. Blood flow in the fingers is 33±10 mL/g/min at 20° C. while in the leg, forearm, and abdomen the blood flow is 4 to 6 mL/g/min at 19 to 22° C. The physiology is consistent with oxygen saturation being diminished in poorly perfused volumes or regions of the body. The physiology is also consistent with the observed differences in localized blood glucose concentration. When glucose concentrations vary rapidly, a difference develops throughout the body in local blood glucose concentrations as a result of differences in local tissue perfusion. For example, the blood flow in the fingers of the hand is greater than in alternative sites. This means that the blood glucose concentration in the fingertips equilibrates more rapidly with venous blood glucose concentration. Furthermore, the magnitude of differences in local glucose concentrations between two sites is related to the rate of change in blood glucose concentrations. Conversely, under steady-state conditions, the glucose concentration throughout the body tends to be uniform.

The following physiological interpretations are deduced from these studies. First, during times of glucose concentration change, the glucose concentration of the outer tissues of the arm lag behind that of the fingertip. Second, a well-recognized difference between the fingertip and the forearm is the rate of blood flow. Third, differences in circulatory physiology of the off-finger test sites lead to differences in the measured blood glucose concentration. Fourth, on average, the arm and finger glucose concentrations are the same, but the correlation is not one-to-one. This suggests differences between traditional invasive glucose concentrations and alternative invasive glucose concentrations are different during time periods of fasting and after glucose ingestion. Fifth, the relationship of forearm and thigh glucose concentrations to finger glucose concentrations is affected by proximity to a meal. Meter forearm and thigh results during the 60 and 90 minute testing sessions are consistently lower than the corresponding finger results. Sixth, differences are inversely related to the direction of blood glucose concentration change. Seventh, rapid changes in glucose concentration produce significant differences in blood glucose concentrations measured at the fingertip and forearm. Eighth, for individuals, the relationship between forearm and finger blood glucose concentration is more consistent than the relationship between individuals. However, the magnitude of the day-to-day differences has been found to vary. Finally, interstitial fluid (ISF) leads as opposed to lag plasma glucose concentration in the case of falling glucose concentrations due to exercise or glucose uptake due to insulin. Corresponding differences in oxygen concentration are indicated by the physiological differences described. One method of increasing the localized perfusion to minimize these differences is photostimulation, as described herein

Instrumentation

The photostimulation instrumentation is either separate from the noninvasive analyzer or is integrated into the noninvasive analyte property analyzer. In either the integrated or non-integrated case, the photostimulation apparatus preferably uses a first light emitting source and the noninvasive analyzer uses a second light emitting source. For example, the photostimulation apparatus uses a first light emitting source, such as a light emitting diode, while the noninvasive analyte property analyzer preferably uses a second photonic source, such as a broadband source or a second light emitting diode. Preferably, the photostimulation instrumentation uses a source or sources that operate over spectral regions used to stimulate perfusion, such as about 890 or 910 nm, while the noninvasive analyzer uses a source that operates over a second spectral region used to analyze the blood analyte property, such as a source or source filter combination including wavelengths longer than about 1100 nm for a noninvasive glucose concentration estimation. Preferably, the photostimulation source uses a source having stronger photon power density and the noninvasive analyzer uses a broadband source with smaller power density at the wavelengths used by the photostimulation source. The broadband source for the noninvasive analyzer allows use of multivariate regression with multiple wavelengths, such as about 10, 20, 50, 100 or hundreds of wavelengths. Preferably, the photostimulation source is used at a first time and the noninvasive analyzer source is used at a time after the use of the photostimulation source. Preferably, the photostimulation source or sources is in a first housing and the noninvasive analyzer source is in a second housing.

Instrumentation for noninvasive analysis is dependent upon the analyte property to be determined. For example, a pulse oximeter has different components than that of a noninvasive glucose concentration analyzer. Instrumentation is described, infra, for only the noninvasive glucose concentration analyzer. Components of the described noninvasive analyzers have corresponding parts in a pulse oximeter and in other noninvasive analyzers. For example, both a noninvasive glucose concentration analyzer and a pulse oximeter have a source, optics, and detector. The particular instrumentation described, infra, for a noninvasive glucose concentration analyzer is exemplary in nature and is not intended to limit the scope of possible noninvasive analyzers used according to the invention.

A spectrophotometric based noninvasive glucose concentration analyzer includes at least: a source, light directing optics, a sample interface, at least one detector, and a data analysis algorithm. The invention includes at least permutations and combinations of photon based noninvasive analyzers described herein.

Noninvasive glucose concentration estimation using a near-infrared analyzer generally involves the illumination of a small region on the body with near-infrared electromagnetic radiation, such as light in the wavelength range about 700 to 2500 nm or ranges therein, such as about 1100 to 2500 nm, 1100 to 1800 nm, or 1150 to 1850 nm. Incident light is partially absorbed and partially scattered according to its interaction with the constituents of the tissue prior to being transmitted or diffusely reflected to a detector. The detected light contains quantitative information that corresponds to the interaction of the incident light with components of the body tissue, such as water, fat, protein, hemoglobin, and glucose. Accordingly, any analyte sufficiently absorbing or scattering the noninvasive analyzers probing light is a potential analyte for determination of an analyte property.

A noninvasive glucose concentration analyzer has one or more optical paths from a source to a detector. Light sources include any of: a blackbody source, a tungsten-halogen source, one or more light emitting diodes (LEDs), or one or more laser diodes. For multi-wavelength spectrometers, a wavelength selection device is optionally used or a series of optical filters is used for wavelength selection. Wavelength selection devices include any of: one or more gratings, prisms, and wavelength selective filters. However, variation of the source, such as varying which LED or diode is firing, is optionally used. Detectors are in the form of one or more single element detectors or one or more arrays or bundles of detectors, such as indium gallium arsenide (InGaAs), lead sulfide (PbS), lead selenide (PbSe), silicon (Si), mercury cadmium telluride (MCT), or the like. Detector configurations further include arrays of any of, such as InGaAs, PbS, PbSe, Si, MCT, or the like. Light collection optics, such as fiber optics, lenses, and mirrors are used in various configurations within a spectrometer to direct light from the source to the detector by way of a sample. In addition, a detector array optionally has a single multiplexor, internal and/or localized electromagnetic interference shielding, and/or at least one uncorrected channel for common mode correction.

Calibration

Noninvasive analyzers require calibration. This is true for all types of noninvasive analyzers, including: pulse oximeters, glucose concentration analyzers, and traditional invasive, alternative invasive, noninvasive, and implantable analyzers. One fact associated with noninvasive glucose concentration analyzers is the fact that they are secondary in nature, that is, they do not measure blood analyte properties directly. This means that a primary method is required to calibrate these devices to measure the blood analyte property properly. Many methods of calibration exist. Calibration methods for noninvasive glucose concentration analyzers are provided, infra, which exemplify calibration methods for noninvasive analyzers.

In yet another embodiment of the invention, calibrations are built using blood samples collected from well perfused regions of the body, such as an artery, vein, fingertip, or toe. It is an object of this invention to reduce sampling error in a subsequent measurement phase by photostimulating a sample site, such as a body part that is not an artery, vein, fingertip, or toe to enhance localized perfusion of the body part thereby reducing or eliminating systemic differences in sampling resulting from sampling blood or tissue that is different in terms of blood constituent properties as a result of being in a region that is well perfused for calibration and less well perfused for subsequent measurements.

In still yet another embodiment of the invention, a calibration is built using samples drawn from a region that is not well perfused. However, the samples for calibration are collected after the not well perfused region is treated with photostimulation to enhance the localized perfusion. Subsequent prediction samples are similarly drawn from regions of the body that are not well perfused after photostimulation has increased the localized perfusion.

Calibration of Noninvasive Glucose Concentration Meters

One noninvasive technology, near-infrared spectroscopy, provides the opportunity for both frequent and painless noninvasive measurement of glucose concentration. This approach involves the illumination of a spot on the body with near-infrared (NIR) electromagnetic radiation, light in the wavelength range about 750 to 2500 nm or regions therein, such as about 1100 to 2500 nm. The light is partially absorbed and scattered, according to its interaction with the constituents of the tissue. The actual tissue volume that is sampled is the portion of irradiated tissue from which light is transflected or diffusely transmitted to the spectrometer detection system. With near-infrared spectroscopy, a mathematical relationship between an in-vivo near-infrared measurement and the actual blood glucose concentration needs to be developed. This is achieved through the collection of in-vivo near-infrared measurements with corresponding blood glucose concentrations that have been obtained directly through the use of traditional invasive measurement tools or any appropriate and accurate traditional invasive reference device. The calibration data is collected with calibration subjects. The calibration is subsequently applied to spectral data collected from measurement subjects. A measurement subject is optionally a member of the calibration subjects. Measurement subjects are also referred to as prediction subjects.

For spectrophotometric based analyzers, there are several univariate and multivariate methods used to develop this mathematical relationship. Herein, multivariate methods refer to methods using at least ten different wavelengths. The basic equation which is being solved is known as the Beer-Lambert Law. This law states that the strength of an absorbance/reflectance measurement is proportional to the concentration of the analyte which is being measured as in equation 3,
A=ε b C eq. 3
where the absorbance/reflectance, A, measurement is at a given wavelength of light, the molar absorptivity, ε, associated with the molecule of interest at the same given wavelength, b is the distance that the light travels, and C is the concentration or saturation of the molecule of interest, such as oxygen, hemoglobin, or glucose.

Chemometric calibrations techniques extract the glucose signal from the measured spectrum through various methods of signal processing and calibration including one or more mathematical models. The models are developed through the process of calibration on the basis of an exemplary set of spectral measurements known as the calibration set and associated set of reference blood glucose concentrations. Multivariate models requiring an exemplary reference glucose concentration vector for each sample spectrum are preferably used, such as partial least squares (PLS) and principal component regression (PCR).

Because every method has error, it is necessary that the primary device used to measure blood glucose concentration is as accurate as possible to minimize the error that propagates through the developed mathematical relationship. In one embodiment, photostimulation is used to reduce errors in the analyte properties used to build the mathematical relationship or model.

The difference between alternative site glucose concentrations and traditional site glucose concentrations introduces errors associated with sampling into alternative site glucose concentration analyzers. Stated again, a calibration built using well perfused blood samples will often result in erroneous predicted analyte properties, such as a glucose concentration, when operating on a region that is not well perfused. These differences in glucose concentration are reduced by pretreating or concurrently with measurement treating the tissue sample site with photostimulation to enhance vasodilation and localized perfusion. Several examples illustrative of photostimulation in combination with a noninvasive analysis follow.

### EXAMPLE II

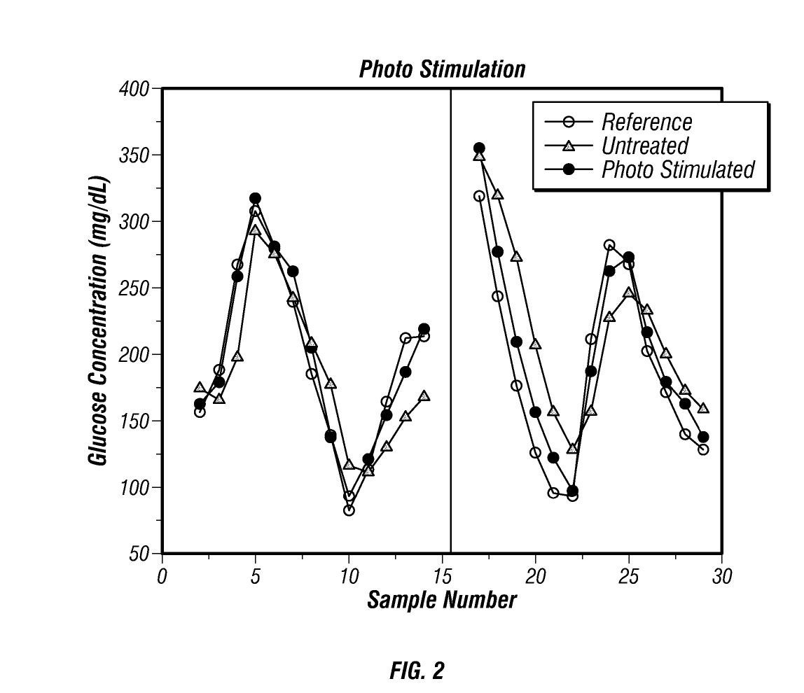

Referring now to FIG. 2, a graph is presented which shows an example of photonic-stimulation used to reduce or eliminate the differences in the glucose concentration between the alternative sampling site of the forearm and the traditional sampling site of the fingertip in terms of dampening and lag. In this study, a number of subjects were run through glucose concentration excursions driven by the combined use of carbohydrate intake and insulin injections. In this study, one forearm site was pretreated with 890 nm photostimulation while the contralateral site on the opposite forearm and fingertips were left untreated. The 890 nm stimulation was performed with three 890 nm LEDs for a period of 30 minutes immediately prior to the glucose concentration data collection. Invasive glucose concentration determinations were subsequently obtained every 20 minutes from all three locations. For two representative subjects, the resulting glucose concentration profiles are presented in FIG. 2. In the first case, the photo-stimulated site is observed to have a higher correlation with the fingertip reference glucose concentration compared to the untreated site. Both the dampening and lag observed in the untreated forearm versus the fingertip are not observed in the glucose concentration profile obtained from the photo-stimulated site. This indicates that the photo-stimulated site is better perfused. In the second presented example, the dampening and lag of the photo-stimulated site is observed to be less pronounced than compared to the untreated site. However, some lag is still initially observed. Subsequently, better optical coupling techniques were used that reduced the percentage of subjects that showed a lag. The reduction in variation via photostimulation between the well perfused fingertips and less well perfused arm results in a sample volume on the arm for a noninvasive analyzer that yields more accurate blood analyte property determinations using a calibration developed using well perfused blood samples.

The increased perfusion that results in the alternative site glucose concentrations more closely tracking the traditional site, such as a fingertip, glucose concentrations is important for several reasons. Medical professionals and diabetes educators have been trained for a generation on the treatment of diabetes with the use of arterial or fingertip glucose concentrations. A large body of literature and indeed medical practice is based upon traditional site glucose concentration determinations. A systematic difference between the body sites will lead to a systematic bias in treatment of diabetes by these educators until medical practice is altered. While the Food and Drug Administration has allowed manufacture, sale, and use of glucose concentration determination methods and apparatus for alternative site glucose concentration determination, they have separate labeling requirements in terms of testing during stable glucose concentration periods and not relying on alternative site glucose concentration determination for timely detection of hypoglycemia. The large number of glucose concentration equalization approaches by large companies, such as heating, partial vacuum, and rubbing of the sample site as outlined above is further evidence of the importance of an equalization approach. Further, an error calculation of a medical device of a well perfused and/or equalized sample alternative sampling site versus a traditional site fingerstick reference has have better accuracy and precision compared to an untreated alternative site glucose concentration error calculation versus a traditional fingertip reference method.

### EXAMPLE III

A photonic-stimulation device or apparatus is used as a stand alone device or alternatively is incorporated into a more complex apparatus, such as a part of a noninvasive analyzer. In two additional embodiments, the photostimulation device is used alone, in the invasive glucose concentration determination section, or as part of a larger device, in the noninvasive glucose concentration determination section.

Source or Illumination Optics

A general overview of a photonic-stimulation source with some possible embodiments follows in this section. A photostimulation apparatus includes at least: a power supply and a source. A wide number of sources are available as light stimulation sources. These include but are not limited to: light emitting diodes, broadband sources, lasers, and diode lasers.

A preferred photostimulation source is a light emitting diode (LED) or multiple light emitting diodes over a narrow wavelength range, such as a wavelength range about 100 nm wide. The source preferably is projected onto, at, or near a sample site. As detailed, supra, stimulation at 890 or 910 nm results in release of nitric oxide. A broader wavelength range is alternatively used to stimulate the same release. The literature shows that the excitation group of interest is a sulfylhydryl group. Additional literature indicates that absorbance of the light by deoxyhemoglobin that is coordinated with the heme group results in the release of nitric oxide. Therefore, the broader potential range of photonic-stimulation is all regions having hemoglobin or the sulfylhydrl groups absorbance. As the absorbance of the agents responsible for the release of nitric oxide decreases the efficiency of coupling the light into the release of nitric oxide decreases. Therefore, wavelengths near the peak absorbances of the coupling molecular structures are preferable. For example, for deoxyhemoglobin light stimulation in regions about 890 or 910 nm is preferably. However, wider regions of light stimulation are possible, such as 850 to 950 nm or less preferably 700 to 1000 nm. Broader spectral ranges are alternatively used with decreasing efficiency. In summary, it is desirable to excite any molecular structure that has the net result of achieving increased perfusion of a sample site through the mechanism of photostimulation.

As the photonic-stimulation process occurs, there is possible ancillary heating due to the physical processes associated with absorbance. However, the photonic-stimulation process described herein stimulates a secondary action beyond heating to induce enhanced perfusion. This distinguishes the process from heating of the sample site for increased perfusion as taught by others, such as Robinson, U.S. Pat. No. 6,152,876 and Rohrscheib, U.S. Pat. No. 6,240,306.

Several examples of photonic illumination systems are illustrated by way of example, infra.

### EXAMPLE IV

In yet another example of the invention, broadband light is used to perform photostimulation. This is a less preferred method as many of the wavelengths of a blackbody source do not induce nitric oxide release after molecular absorbance. In addition, radiative heating of the tissue with a broader range source is not desirable in the mechanism of photonic-stimulation. However, the radiative heat from the source absorbed by the sample results in heating of the tissue and dilation of capillaries increasing perfusion. Thus, the synergistic approach of photonic heating in combination with radiative heating is an alternative method on increasing local perfusion. In addition, radiative heating is optionally used to stabilize the tissue sample site, which aids in precision of collected noninvasive spectra.

It is noted that undue heating of the sample site has its costs. First, a large amount of the broadband light is not inducing nitric oxide release. This makes the system less efficient. For example, a larger source and/or power supply is required. Second, it is well known that undue heating of the sample results in many near-infrared absorbance bands to change in a nonlinear fashion. This greatly complicates subsequent analyses, particularly those based upon soft models or multivariate models.

### EXAMPLE V

In still yet another example of the invention, an additional source combination for photonic-stimulation is use of a broadband source in conjunction with optical filters. Optical filters used to isolate one or more spectral regions include: longpass, shortpass, or bandpass filters. This system allow one or more wavelength regions of interest to penetrate into the sample. Further, this system allows use of broadband sources that are relatively inexpensive. The same broadband source is optionally used as the light source for the noninvasive analyzer. Baffles, heat sinks, cooling fins, and the like are preferably used to dissipate excess heat.

### EXAMPLE VI

In still yet another example of the invention, alternative photonic-stimulation sources include: lasers and laser diodes. Typically these sources deliver a larger magnitude of photons. This allows a more rapid stimulation and subsequently a more rapid increase in perfusion. However, these devices are typically larger and more expensive. They are optionally used according to this invention, but the LED's are preferred.

### EXAMPLE VII

In an additional example of the invention, photonic-stimulation sources are configured as individual elements, as multiple elements, or as an array. In a first case, a single 910 nm LED is used for photostimulation. In alternative cases, two or more LED's are used for excitation at the expense of greater power consumption, but with the benefit of a shorter illumination time.

### EXAMPLE VIII

In another example, a number of LED's, such as about three, are placed into a guide element as discussed below. This combination allows photonic-stimulation directed with precision at the sample site by the guide prior to subsequent noninvasive measurements. Further, this mechanism frees the user to perform additional actions. Optionally, the guide is used in combination with a coupling fluid, such as a fluoropolymer, a fluorocompound, Fluorinert, FC-40, FC-70, or equivalent.

### EXAMPLE IX

In yet another case, photostimulation is performed using an array of LED's. In this case a larger tissue sample is photo-stimulated, more light intensity is delivered at or about a given tissue sample site and/or required illumination time is reduced. For example, a patch with an m by n array where m and n are positive integers, a geometric pattern, or a random pattern of LED's is used to cover a larger surface area of the sample.

### EXAMPLE X

In another example of the invention, more than one range of wavelengths is alternatively used in the illumination source. For example, two or more types of LED's are used in the source. This results in a wider wavelength range of incident photons or two or more bands of incident photons. This method allows direct targeting of two or more molecular species having two or more absorbance features that lead to dilation of capillaries based upon photostimulation. In addition, broader coverage of a given absorbance band is achieved in terms of wavelengths using more than one LED type.

Permutations and combinations of the source illumination systems described, supra, are also aspects of this invention. For example, a mixture of species of illumination elements is used or and LED is used in combination with a broadband source.

Sample Interface

The interface of the photonic source to the sample is of particular concern.

Alignment

The accuracy and/or precision of the incident photons relative to the sampling site is important. For example, the increased perfusion due to the incident photons is limited in surface area and its associated volume. Generally, sampling in the perfused region is desirable. Embodiments where sampling outside of the perfused region is desirable are described in the alternative embodiments below. Many possibilities exist for sampling where the perfusion is enhanced; a few examples are described below.

### EXAMPLE XI

In still yet another example of the invention, a method of sampling where the perfusion is enhanced is by visually aligning sampling to be where the photons were incident upon the skin. This is performed in a number of ways, such as by memory; spatially relative to one or more sample features, such as a joint or a freckle; or by measurement.

### EXAMPLE XII

Another method of sampling where the perfusion is enhanced is by using a larger illumination area. For example, a diffusing optic or an array of illuminators is used. Control of the illumination intensity distribution is important for calibration transfer. For example, distributing light evenly over a range of radii from a detection fiber allow many depths of tissue sample to be analyzed in a calibration. Subsequent measurements at similar depth due to similar illumination patterns are easier to predict with.

### EXAMPLE XIII

In yet another method of sampling, perfusion is enhanced by the use of a guide. Guides are described in detail below. Generally, a guide is a replaceably attached apparatus used as one-half of a lock an key mechanism. One use of a guide is the alignment of the incident photons relative to the sampling site and/or the alignment of a sensor or probing device relative to the same sampling site. The guide ensures that the illumination optics for the photostimulation and/or noninvasive measurement is accurately and precisely directed at a targeted sample tissue surface area and volume.

Method

In the simplest embodiment of this invention, photostimulation is performed prior to and/or during sampling at and/or about the targeted tissue sample surface area and optically sampled tissue volume.

Timing

The relative timing of photostimulation and sampling is dependent upon the application. Specific examples of duty cycles and timing relative to sampling are provided in the preferred and alternative embodiments. Several illustrative examples of timing of photostimulation follow.

### EXAMPLE XIV

In some instances a photo-stimulator is not optically attached to the sample site when not in use. In these cases the source is either manually turned on has an automatic activation means. For example, application is induced by pressure applied when sampling, by a switch mechanism in a guide, by sensing movement, or by proximity to a magnetic field. Once activated, the duty cycle is pulsed, continuous, or semi-continuous. Photostimulation duration is either under manual control or is under automated control, such as being deactivated after a preset time interval. Photostimulation is optionally performed at times including any of: a beginning of a day or operating period, prior to sampling by multiple minutes, just prior to sampling, during sampling, or for a period of time within about 1, 2, or 4 hours from a time of subsequent determination of the analyte property.

### EXAMPLE XV

If the photo-stimulator is optically attached to the sample site, the duty cycle is continuous, semi-continuous, or manually activated by the user. For example, a light emitting diode based photo-stimulator is optionally installed into a guide element. The stimulator is programmed to turn on at a given time of day, continuously illuminate, have a duty cycle, or have manual activation means.

### ILLUSTRATIVE EMBODIMENTS

In a another embodiment of the invention, photostimulation is used in conjunction with oxygen saturation determination or estimation. More particularly, photostimulation at or near a sample site is used to enhance perfusion of the sample site, such that the blood or tissue concentration of oxygen and hemoglobin more accurately tracks that of arterial, venous, fingertip, or well perfused body sites. Photostimulation and pulse oximetry are performed as described throughout this specification.

In yet another embodiment of the invention, photostimulation is used in conjunction with noninvasive glucose sampling and/or measurement techniques. More particularly, photostimulation at or near a sample site is used to enhance perfusion of the sample site, such that the blood or tissue concentration of glucose more accurately tracks that of arterial, venous, fingertip, or well perfused body site glucose concentration. Photostimulation, glucose sampling, and glucose concentration measurement techniques are performed as described throughout this specification. The glucose concentration determinations are invasive, minimally invasive, or noninvasive. The invasive glucose concentration determinations are preferably at alternative sites; however, traditional sites are alternatively used. Several species or examples of this embodiment are described below.

### EXAMPLE XVI

In still yet another example of the invention, photostimulation is used in conjunction with noninvasive estimation of glucose concentration. More particularly, photostimulation at or near a sample site is used to enhance perfusion of the sample site such that the blood or tissue concentration of glucose more accurately tracks that of arterial, venous, fingertip, or well perfused body site glucose concentration. A photonic-stimulator is used in combination with a noninvasive glucose concentration analyzer to generate glucose concentration determinations from at least one subject. The noninvasive analyzer includes: a source, a sample, light direction optics, and at least one detector. The analyzer preprocesses the data and uses multivariate analysis in the glucose concentration determination.

### EXAMPLE XVII

In an additional example, a noninvasive glucose concentration analyzer is used in combination with photonic-stimulation. The photonic-stimulator is packaged in a plug that couples into a guide. A guide is replaceably attached to a coupling optic of the noninvasive analyzer. The guide is also replaceably attached to a sample site, such as a subject's forearm. The plug contains at least one 890 nm LED run off of a battery that is used to photo-stimulate the sample site at least prior to the first glucose concentration determination of a day. The glucose concentration analyzer includes: a tungsten halogen source, an optional backreflector, and at least one optical filter prior to the sample. The optical filter is used as a heat blocker and/or as an wavelength order sorter. The preferred embodiment further has the step of directing the incident light onto a sample, preferably the dorsal aspect of the forearm using the guide. Photons are Is collected from the sample and are directed to a grating and subsequently to at least one detector. The spectral range is about 1100 to 2450 nm or ranges therein. Preprocessing is performed on the spectra. Forms of at least one of averaging, smoothing, taking the n^{th} derivative where n is a positive integer, clustering, performing multivariate analysis, and mean centering are performed. Finally, an estimated glucose concentration is generated.

### EXAMPLE XVIII

In another example, a noninvasive glucose concentration analyzer is used in combination with photonic-stimulation. The photonic-stimulator is packaged in a plug that couples into a guide. The plug contains a single element 890 nm LED run off of a battery that is used to photo-stimulate the sample site on the day of a subsequent noninvasive measurement at least prior to the first glucose concentration determination of a data collection period, such as for an individual sample, for about six hours, or about a day. The glucose concentration analyzer includes: a source, such as a tungsten halogen source of less than five Watts, a backreflector, and at least two optical filters prior to the sample. At least one of the optical filters is used as a heat blocker or as an order sorter. This embodiment directs the incident light onto a sample, such as an alternative site through the use of a guide. Diffusely reflected photons are collected from the sample into at least one fiber optic and are directed to a grating and subsequently to an array detector. The spectral range is about 1150 to 1800 nm or ranges therein. Preprocessing is performed on the spectra. Forms of at least one of averaging, smoothing, taking the nth derivative where n is a positive integer, clustering, performing multivariate analysis, and mean centering are performed. A glucose concentration is estimated from the resulting noninvasive spectra.

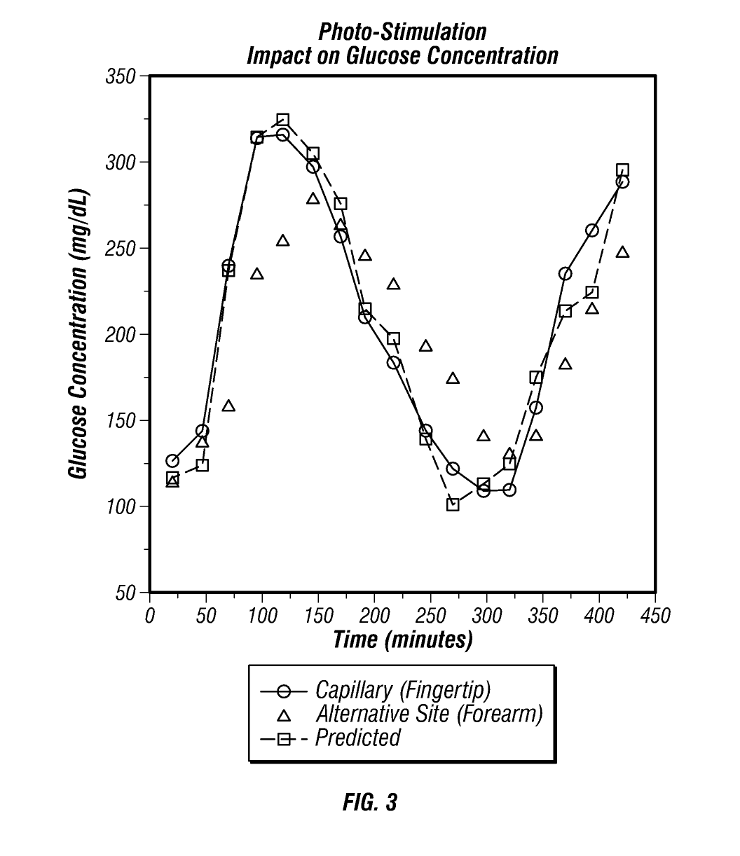

Referring now to FIG. 3, glucose profiles using the above described noninvasive analyzer are presented. This glucose concentration profile is that of a single subject. The glucose concentration rises were induced through carbohydrate intake to create a large glucose concentration test range. Insulin was used to bring the glucose concentrations down to validate model performance as predicting on the glucose signal instead of an ancillary correlation. Carbohydrates were subsequently ingested in order to further test the model by breaking remaining correlations between glucose concentration and ancillary interferences. Noninvasive glucose concentration determinations were performed approximately every twenty to twenty-five minutes as were traditional fingertip glucose concentration determinations and alternative site glucose concentration determinations from a site on the forearm that was not treated. Clearly, the noninvasive glucose concentration estimations track the reference glucose concentrations. Of note, the predicted glucose concentrations from the photo-stimulated site track the fingertip reference glucose concentrations more accurately than the alternative site forearm reference glucose concentrations.

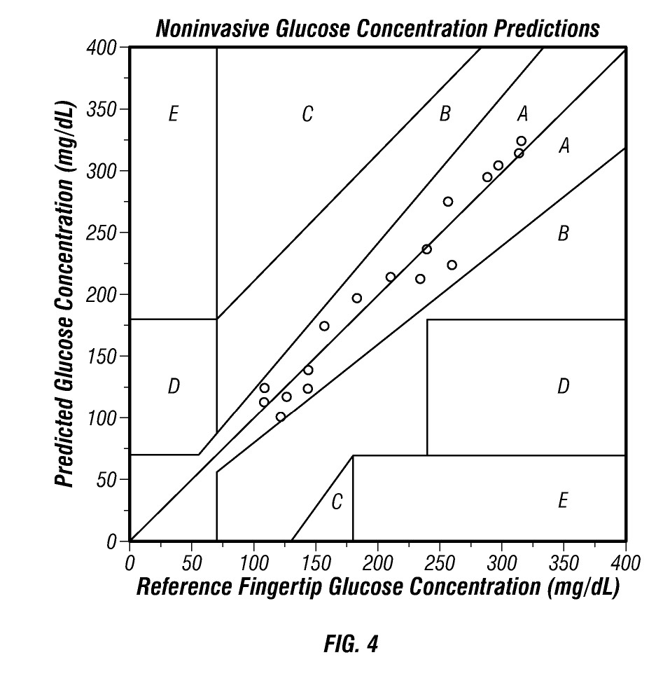

Referring now to FIG. 4, a graph shows the noninvasive glucose concentration estimations and fingertip reference glucose concentrations from FIG. 3 plotted in a concentration correlation plot ovelaid with a traditional Clarke error grid. In a Clarke error grid, all points in the ‘A’ and ‘B’ region are clinically acceptable with the points in the ‘A’ region having less than twenty percent error. A crude guide to acceptable data is 95% of the points falling into the ‘A’ or ‘B’ region. In this study, 100% of the values fell into the ‘A’ region. The standard error of prediction is 14.6 mg/dL, the R is 0.98, and the F-value is 27.17.

### EXAMPLE XIX

In still yet another example, noninvasive glucose concentration estimations are provided using a noninvasive analyzer according to the invention with and without photonic-stimulation. Sensys Medical, Inc. pilot glucose concentration analyzers were used in this study. The pilot analyzers included: a tungsten halogen source, a backreflector, a silicon window, a guide, a plug fit into the guide, a single fiber optic to collect diffusely reflected light, a slit, a grating, and an array of detectors. In this example, the analyzers were used on a forearm tissue sample site. Critical to the analyzer is the resulting signal-to-noise ratio, stability, and resolution of the analyzer as opposed to the specific elements used.

The guide was configured with a photonic-stimulator attachment. In this case, three 890 nm light emitting diodes were used in the guide and were positioned roughly one millimeter from the sample site surface. A total of six subjects participated in this study. Each subject was treated with photonic-stimulation on one arm over the sampling site and not on the opposite arm for a period of thirty minutes prior to collection of any noninvasive glucose spectrum on a given test day. In this example, photostimulation was performed only prior to the first noninvasive glucose concentration estimation and was not repeated prior to subsequent noninvasive or invasive glucose estimations. Each subject was then run through a glucose concentration excursion lasting for approximately four hours. Reference glucose concentration determinations were collected every twenty minutes from the fingertip and forearm with an invasive glucose concentration analyzer. In addition, noninvasive glucose spectra were collected every twenty minutes from each forearm representing samples from untreated and photonically treated sample sites. One-half of the subjects were treated with photonic-stimulation on their left arm and one-half were treated on their right arm.

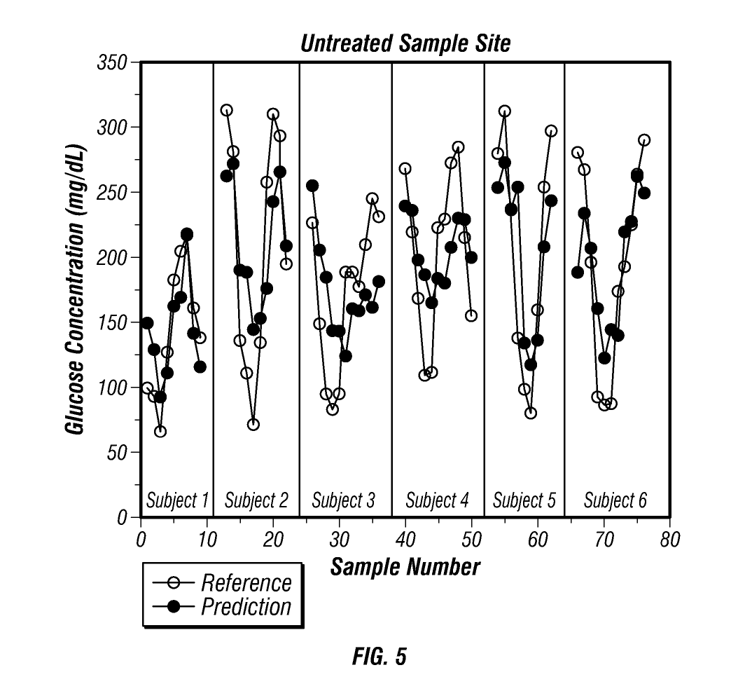

For each of the six subjects, the noninvasive spectra were analyzed with a single calibration model. The model included: a spectral preprocessing routine, an outlier analysis module, and a multivariate analysis module. The spectral range was 1200 to 1800 nm. Referring now to FIG. 5, a graph shows the resulting glucose concentration estimations from the noninvasive spectra collected from the untreated sample site of each of the six individuals overlaid with their corresponding invasive reference glucose determinations. For subjects identified as two through five, the estimated glucose concentrations using the noninvasive analyzer are dampened in their total glucose concentration range relative to the reference glucose concentrations. Subjects one to four and subject six clearly have a predicted glucose concentration profile that lags the reference glucose concentration. This is consistent with a glucose concentration at the sampling site that is not well perfused and results in a glucose concentration profile that is dampened and/or lagged versus a well perfused reference glucose region, such as a fingertip.

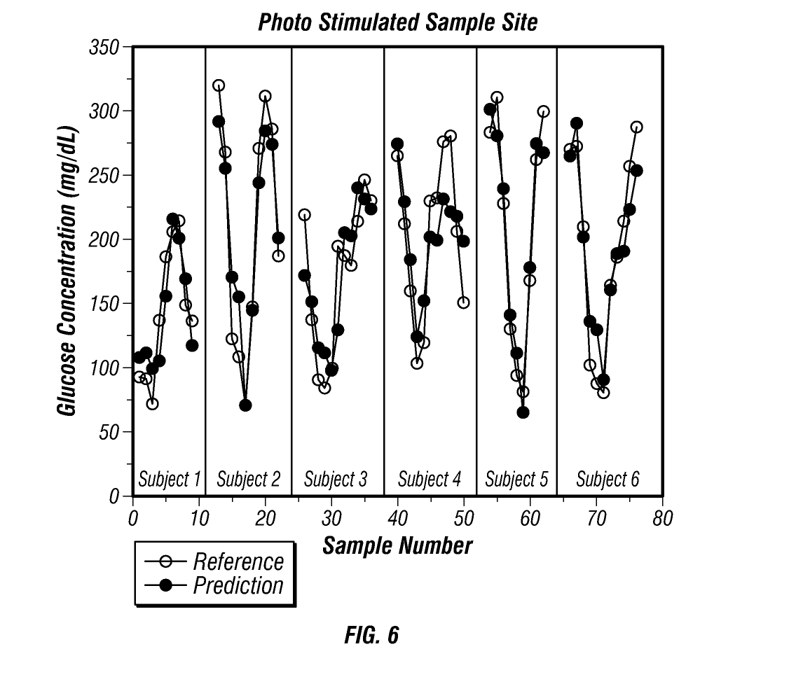

Referring now to FIG. 6, a graph shows the resulting glucose concentration estimations from the noninvasive spectra collected from the treated sample site of each of the six individuals overlaid with their corresponding invasive reference glucose concentration determinations. For subjects one to three, five, and six, the estimated glucose concentration using the noninvasive analyzer closely tracks the reference glucose concentrations. Subject four has an estimated glucose concentration profile that initially tracks and later is dampened versus their corresponding reference glucose concentrations. These results are consistent with the photostimulation treatment of the sampling site equalizing the glucose concentration between the fingertip and the forearm sample site. Further, the equalization persisted in all but one of the subjects over the entire four hour test period.

Photostimulation was observed in the above study to result in equilibration of the glucose concentration between the less well perfused sample site and the well perfused reference site. Again, the photostimulation resulted in vasodilation that led to the equilibration of the glucose concentration in the two body compartments. The noninvasive glucose concentration estimation model was then able to predict more accurately the glucose concentration due to the noninvasive analyzer sampling a region that actually had glucose concentrations that correlate with the reference glucose concentration.

In the above study, photostimulation was performed for thirty minutes with three LED's at the beginning of a testing period. The resulting vasodilation resulted in increased perfusion of the sampling site for a period of hours. Additional data, not presented here, indicates that a single LED results in the same vasodilation results. Therefore, one LED is sufficient to equalize the glucose concentration to the extent that a noninvasive glucose concentration analyzer predicts more accurate glucose concentrations. In addition, the mechanism of vasodilation suggests that photostimulation is optionally performed periodically performed throughout a given day rather than just at the beginning of a day. For example, photostimulation is used before the first sample of the day, with each sample of the day, or at periodic intervals during the day. The duration of stimulation of each interval is optionally varied. For example, the first photostimulation duration of the day is optionally longer than subsequent treatments of the sample site.

### EXAMPLE XX

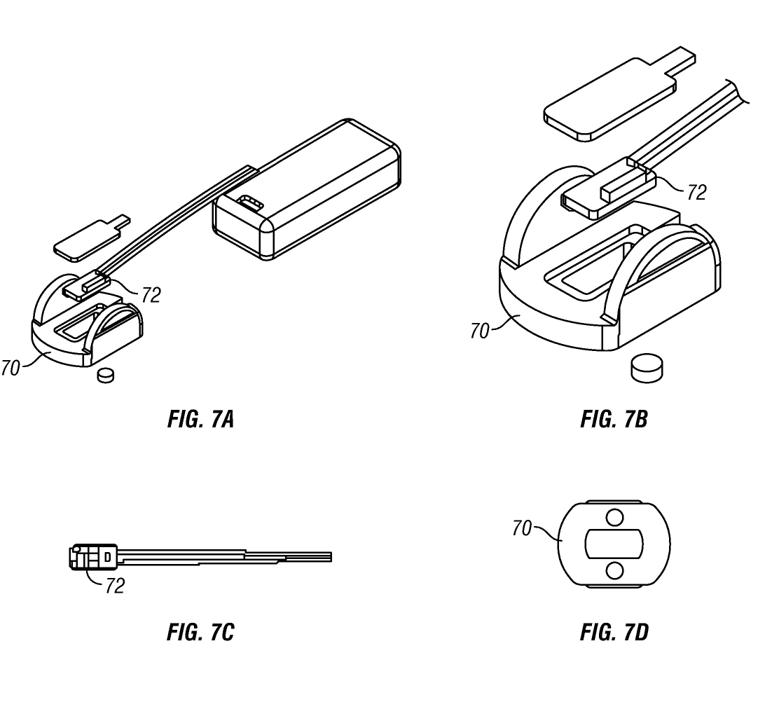

In yet another example, a photonic-stimulator attachment is an attachment coupled to the tissue sampling site via the guide. Referring now to FIGS. 7a-7d, an example of a photonic-stimulator attachment is presented coupled to a guide. In the embodiment pictured, a guide 70 is coupled to a plug 72. The plug contains three LEDs along with a circuit board. Power is supplied via an auxiliary battery or power pack. The power supply is optionally integrated into the plug. In this example, magnets are used to facilitate reproducible alignment between the guide and the plug and hence between the plug containing the LEDs and the sample site.

The photonic-stimulator attachment results in many of the advantages or properties of a plug. The photonic-stimulator attachment is optionally also used as a plug to accomplish at least one of hydration of the sampling site by occlusion, protection of the sampling site from physical perturbation, protection of the sampling site from contamination, alignment of the guide, and allowing an aesthetic appearance, such as a watch, ring, ornamental display, or graphical symbol.

In another embodiment of the invention, photostimulation is used to enhance perfusion at traditional sample sites in combination with noninvasive analysis, such as pulse oximetry saturation or glucose concentration estimation. The technique is beneficial for traditional sampling sites, such as a fingertip, in subjects that have poor circulation, such as diabetics that have poor circulation in their extremities or subjects with conditions resulting in poor circulation, such as hypotension, vasoconstriction, hypothermia, decreased cardiac output, or local vasoconstriction. The enhanced perfusion increases noninvasive analyzer performance, as described supra. Thus, the technique is beneficial for traditional sampling sites in subjects, such as diabetics that have poor circulation in their extremities.

In still another embodiment of the invention, photostimulation is used in combination with at least one additional perfusion enhancement technique followed with a noninvasive analysis of a tissue analyte property or concentration. Additional perfusion enhancement techniques include:
rubbing at or about the same site;
heating at or about the sample site;
intake of L-arginine by the photostimulated subject;
intake of a surface capillary dilating agent, such as niacin;
applying a negative pressure at or about the sample site; and
application of a topical pharmacologic or vasodilating agents, such as nicotinic acid, methyl nicotinamide, minoxidil, nitroglycerin, histamine, menthol, or capsaicin.

As one example, photostimulation is used in conjunction with heating to enhance perfusion of the sample site. In a second example, L-arginine is ingested in the hours, such as about four hours, prior to use of a subsequent noninvasive technique. The combined perfusion enhancement is then followed by noninvasive techniques as described in the preferred embodiments herein.

A benefit of heating the sampling site is dilation of the capillaries to enhance localized circulation and stabilization of the temperature of the sampling site to minimize spectral variation. In one case, a heating element is placed in close proximity to the sampled site. This heating element is optionally controlled with a feedback sensor as taught in Hazen, Ph.D. dissertation, “Noninvasive Glucose Determination in Biological Matrices”, University of Iowa, Department of Chemistry, 1995. In a second case, photonic heating of the tissue sample site is used in combination with photonic-stimulation resulting in the benefits of photonic-stimulation and heating. As described, infra, different wavelengths of light are optionally used to preferentially heat different layers of the sample site. Alternative sources are potentially used for heating, such as a broadband radiative source, a broadband source limited by filters to one or more spectral regions, a glowbar, one or more LEDs, a laser diode, and a laser. For example, a tungsten halogen source is coupled with one or more longpass, shortpass, or bandpass filters to pass light to the sample site with one or more regions.

Hence, while photostimulation is intended to replace equilibration techniques described herein, such as heating, rubbing, and pulling partial vacuums, it is recognized that there are benefits of using photostimulation in combination with these techniques.

In another embodiment of the invention, the photo-stimulator is in a handheld device that is used in conjunction with the noninvasive analyzer. Photostimulation sources for the handheld device are as described elsewhere in this specification. For example, one or more 890 nm LEDs are powered by a battery to provide photons that are delivered to the sample where they are subsequently absorbed leading to increased perfusion of the sample site. The power supply, source, and optional coupling optics are integrated into a handheld illuminator. The device optionally has means for turning the device on or off. The device is used to photo-stimulate prior to and/or during an noninvasive analysis. In additional cases, the handheld device uses a source including any of: one or more LEDs, a broadband source, a broadband sources coupled with longpass, shortpass, or a bandpass optic, a laser, and a diode laser.

In yet another embodiment of the invention, a guide is used in conjunction with photostimulation. The photo-stimulator is optionally incorporated into the guide or is incorporated into an attachment to the guide, such as in one-half of a lock and key guide mechanism. For instance, one or more 910 nm LEDs are incorporated into a plug along with one or more batteries. The plug is replaceably attached to the guide. The guide itself is replaceably attached to or is near the sampling site. The guide provides control of where the photostimulation is hitting the skin tissue. The photo-stimulator is preferably used with a noninvasive analyzer, such as a noninvasive glucose concentration analyzer or with an oxygen analyzer using oxy- and deoxyhemoglobin signals. It is noted that in the case of an invasive or semi-invasive glucose concentration estimation, a guide need not be left on the sampling site for extended periods of time. It is sufficient to place a guide, photo-stimulate in a position relative to the guide, sample in a position stimulated and remove the guide. Typically, in a noninvasive glucose concentration estimation the guide is left on for a series of glucose concentration estimations.

Photo-stimulator Parameters

General consideration of photo-stimulators are provided in this section. First, parameter considerations for the photo-stimulator apparatus and method of use include: power consumption, size, cost, stability, accuracy of alignment to the sample site, precision of alignment to the sample site, and lifetime. Second, as described, supra, the photo-stimulator contains one or more source elements or an array of sources. A photostimulation apparatus is preferably coupled to the sample site with free space, floating, or fixed coupling optics. The photostimulation is configured to run continuously, be activated by a user, to have preset duty cycles, be motion activated, or be activated by means, such as a magnetic field, when placed near the sampling site.

Photostimulation is performed at or near the sampling site. Therefore, if photostimulation is performed at a different time period from when sampling is performed it is beneficial to have locating means such that sampling occurs at or near the photostimulation site. Means described in the noninvasive embodiments are applicable to this situation. For example, locating means, such as direct measurement, memory, distances to sample features, or relative distances to sample features are potentially used. Optionally, a replaceably attached guide is used as described, supra.

Preferable sampling sites include: a forearm, wrist area, upper arm, torso, thigh, and ear. Photostimulation is optionally used prior to traditional glucose concentration analysis or pulse oximetry percent saturation readings, on locations, such as the fingertip, base of thumb, plantar regions, or toes. This is beneficial for individuals with circulation problems where traditional sampling is hindered. In the case of invasive glucose concentration determination, the increased perfusion allows for smaller lancets and shorter penetration depths for adequate blood volume to be collected and/or used.

Photostimulation in combination with invasive glucose concentration estimation methods has a number of advantages. First, the combination allows for more accurate and precise glucose concentration estimations when compared to traditional fingertip glucose concentration determinations. Second, the decreased lag time makes invasive meters more useful in determination of hypoglycemia. Third, the decrease in dampening allows for more accurate determinations of glucose concentration extremes during hyperglycemic periods. Fourth, photostimulation allows for accurate glucose concentration analysis while glucose concentrations are changing rapidly, for example with rates of change in excess of two mg/dL/min.

In still yet another embodiment of the invention, different tissue layers are preferably heated via the mechanism of light absorbance. This results in the expansion of capillaries due to heat at preferable sampling depths without the interferences associated with undue heating at other sample depths. This is possible as some wavelengths penetrate further into the body based upon the scattering and absorbance coefficients of the illuminated site. Therefore, appropriate selection of wavelengths of incident light preferentially absorb and thus heat different skin depths. For example, mid-infrared (2500 to 14,258 nm or 4000 to 700 cm⁻¹) light absorb in the first few microns of the skin surface due to the strong absorbance of water in these wavelength ranges. Combination band light (2000 to 2500 nm) preferentially absorbs in skin resulting in heat at a greater depth of circa 1 to 2 mm. First overtone (1450 to 1950) and second overtone (1100 to 1450) light preferentially absorbs at depths of 1 to 5 and 4 to 10 mm of depth, respectively, due to the absorbance of water. Therapeutic window light penetrates and heat at greater depths but is highly influenced by the scattering properties of the sample. Visible light is highly scattered and results in heating at a large range of depths. Selection of an appropriate range or ranges of wavelengths results in preferential heating at one or more depths.

In an alternative embodiment, differential measurements in terms of photostimulation is performed. More particularly, temporal and/or spatial differential measurements are performed. Differential measurements are often made in spectroscopy in order to enhance a signal-to-noise ratio or determine a difference in state. A temporal differential measurement is made by performing an analysis before, during, and/or after photostimulation. Typically, a baseline reading is performed. In one case, a noninvasive spectrum is obtained. Photostimulation is then performed. A second noninvasive spectrum is then obtained. Chemometric approaches are then used on the two spectra. Typically, these techniques are subtraction or ratio determination in order to remove background information or enhance the analyte signal-to-noise ratio. For example, the signal, precision of estimated concentration of glucose or urea, or the relative percent saturation of oxygen is enhanced via a differential measurement.

Alternatively, differential measurements are used to determine the impact of photostimulation on the tissue samples site. For example, differences in scattering, water content, or oxygenation are determined.

A spatial differential measurement is made by performing an analysis at two sites. One site is treated by photostimulation and the other site is left untreated. Typically, both analyses are performed at the same time or in close time proximity, such as within about a few seconds or minutes. For example, a baseline reading is performed at the untreated site and a sampling reading is performed at the treated site. For example, in spectroscopy the reference spectrum is collected at the untreated site and the sample spectrum is collected at the treated site. Typically these spectra are subtracted from one another or ratioed in order to enhance the signal-to-noise ratio of an analyte, though additional chemometric approaches are alternatively used. For example, the signal-to-noise ratio of glucose, forms of hemoglobin, oxygenation levels, or urea are enhanced.

In yet another embodiment of the invention, photostimulation is used in combination with noninvasive urea, cholesterol, blood gas, oxygen, hemoglobin, deoxyhemoglobin, or pH determination. Noninvasive techniques used for glucose concentration estimation that are described herein are used for noninvasive analyte property determination of additional analytes. Wavelength regions for urea, blood gases, cholesterol, and pH are described in Robinson, U.S. Pat. No. 6,212,424, Thomas, U.S. Pat. No. 5,630,413, Alam, U.S. Pat. No. 5,792,050, Alam, U.S. Pat. No. 6,061,581, and Alam, U.S. Pat. No. 6,073,037.

Duration of Stimulation

In still yet another embodiment of the invention, photostimulation is used to achieve short and/or long term enhanced perfusion at or about a sample site. Photostimulation is determined to enhance perfusion over a range of time periods initiating in a second and lasting for a number of hours, such as about four to six hours. In addition, repeated photostimulation aids in healing and/or maintenance of tissue. Hence, long-term benefits to the tissue sample state in terms of blood flow are achieved. This is sometimes referred to in the art as angiogenesis.

Angiogenesis is the name given to the development of new capillaries from pre-existing blood vessels. Stimulated endothelial cells form capillary sprouts, which expand and undergo morphogenesis in order to form a mature capillary. Newly formed capillaries then go through a process of proliferation, migration, and invasion into the surrounding tissue to create a fresh network of blood vessels. Angiogenesis occurs normally in the human body during times of development and growth. The developing child in a mother's womb undergoes the creation of a vast network of arteries, veins, and capillaries. Adults, though less frequent, also experience proliferation of new blood vessels. In women, angiogenesis is active during the menstrual cycle each month as new blood vessels form in the lining of the uterus. People with cardiovascular disease often experience angiogenesis as new vessels form around a blocked or diseased vessel. Angiogenesis is also a necessary part of repairing and regenerating new tissue during wound healing.

The extent of angiogenesis is determined by the balance between pro-angiogenic factors and anti-angiogenic factors. One important pro-angiogenic factor involves an increase in the concentration of endogenous nitric oxide. Mechanisms to increase nitric oxide include: exercise; supplementation with L-arginine; supplementation with antioxidants; use of certain drugs, such as statins and estrogen replacements; and the application of light as discussed herein. It is known that illumination of the tissue releases nitric oxide from hemoglobin in red blood cells. The release is local, limiting the effect in other portions of the body.

In one aspect of the invention, photostimulation is used to stimulate and maintain a localized long term increased perfusion effect, such as an angiogenic effect, at the measurement site. Once the area has generated new vasculature, the increased blood flow with the benefits of increased perfusion described herein. In one case, angiogenesis or long term circulation enhancement is used in combination with a noninvasive tissue sample site analyzer. In a second case, angiogenesis or long term circulation enhancement is used in combination with an implantable analyzer, such as an implantable pancreas or an implantable glucose concentration analyzer.

In one embodiment of the invention, angiogenesis of a measurement subject's sample site is induced with repeated use of photostimulation of a sample site on the subject over a period of days. Preferably, the photostimulation is repeated over a period of days prior to noninvasive measurement of the sample site using a noninvasive analyzer or invasive technique to determine an analyte property of the subject, such as a glucose concentration.

In another embodiment of the invention, induced angiogenesis, short term enhanced perfusion, and/or long term enhanced perfusion as described herein is used in combination with an implantable sensor that wirelessly transmits a blood glucose concentration reading to a receiver, such as a handheld receiver. The implantable sensor is preferably placed under the skin, such as in the abdomen, and has an operation period of about weeks, months, about a year, or about three years. Photostimulation is preferably used just before, during, and/or in the thirty days following implantation of the sensor. Optionally, photostimulation is used prior to or during readings after successful implantation. Increasing short and long term perfusion by via photostimulation and/or intake of a surface capillary dilating agent, such as L-arginine or niacin, increases perfusion about the implantable and yields increased signal-to-noise ratios in subsequent readings due to increased blood flow to the sensor and minimizes risks of autoimmune rejection of the implantable. An example of a long term implantable glucose concentration analyzer usable in combination with enhanced perfusion techniques as taught herein is the DexCom™ glucose concentration analyzer (DexCom, Inc., San Diego, Calif.). Methods and apparatus describing an implantable glucose concentration analyzer include Shults, U.S. Pat. No. 6,862,465 and Goode, U.S. Pat. No. 6,931,327, which are both incorporated herein in their entirety by this reference thereto.

Sampling

A noninvasive analyzer as described herein is used for sampling. The noninvasive analyzer includes an integrated, connected, or separate sample probe for sampling a tissue sample site. Optionally, at least part of the sample probe is movable with respect to the sample. Having a sample probe head that is movable relative to the sample enables the sample probe to arrive to close proximity to a sample site, to touch a sample site, or to minimally perturb a sample site.

Coordinate System

Herein, an x, y, and z coordinate system relative to a given body part is defined. A rectangular Cartesian coordinated system having axis designators x, y, and z is used to define the sample site, movement of objects about the sample site, changes in the sample site, and physical interactions with the sample site. The x-axis is defined along the length of a body part and the y-axis is defined across the body part. As an illustrative example using a sample site on the forearm, the x-axis runs between the elbow and the wrist and the y-axis runs across the axis of the forearm. Similarly, for a sample site on a digit of the hand, the x-axis runs between the base and tip of the digit and the y-axis runs across the digit. Together, the x,y plane tangentially touches the skin surface, such as at a sample site. The z-axis is defined as orthogonal to the plane defined by the x- and y-axis. For example, a sample site on the forearm is defined by an x,y plane tangential to the sample site. An object, such as a sample probe, moving along an axis perpendicular to the x,y plane is moving along the z-axis. Rotation or tilt of an object about one or a combination of axis is further used to define the orientation of an object, such as a sample probe, relative to the sample site.

Z-Axis Sample Probe Movement

Control of a noninvasive sample probe along a z-axis is described in U.S. patent application Ser. No. 11/117,104, which is incorporated herein in its entirety by this reference thereto.

In one embodiment of the invention, a noninvasive analyzer sample probe or sampling probe applies a controlled displacement of the sample probe relative to a sample. One or more displaced elements of a sample module are controlled along a z-axis perpendicular to the x,y plane tangential to the surface of the sampled site. The z-axis control of the displaced sample probe element of the sample module provides for collection of noninvasive spectra with a given displacement or no displacement of a tissue sample and for collection of noninvasive spectra with varying applied displacement positions of the sample probe relative to the nominal plane of the sample tissue surface.

Sample probe movement is optionally controlled with an algorithm. In one embodiment, the algorithm uses features extracted from noninvasive spectra and control parameters to direct movement of the sample probe relative to the tissue sample. A feature is any derivative of a spectrum processed to enhance a particular quality that is beneficial to control. A feature is extracted information for purpose of control. Extraction of a feature typically reduces interference that is detrimental to probe movement control. Examples of feature extraction techniques include use of a derivative, a multivariate analyze, or the analysis of intensity spectra for chemical or physical signal.

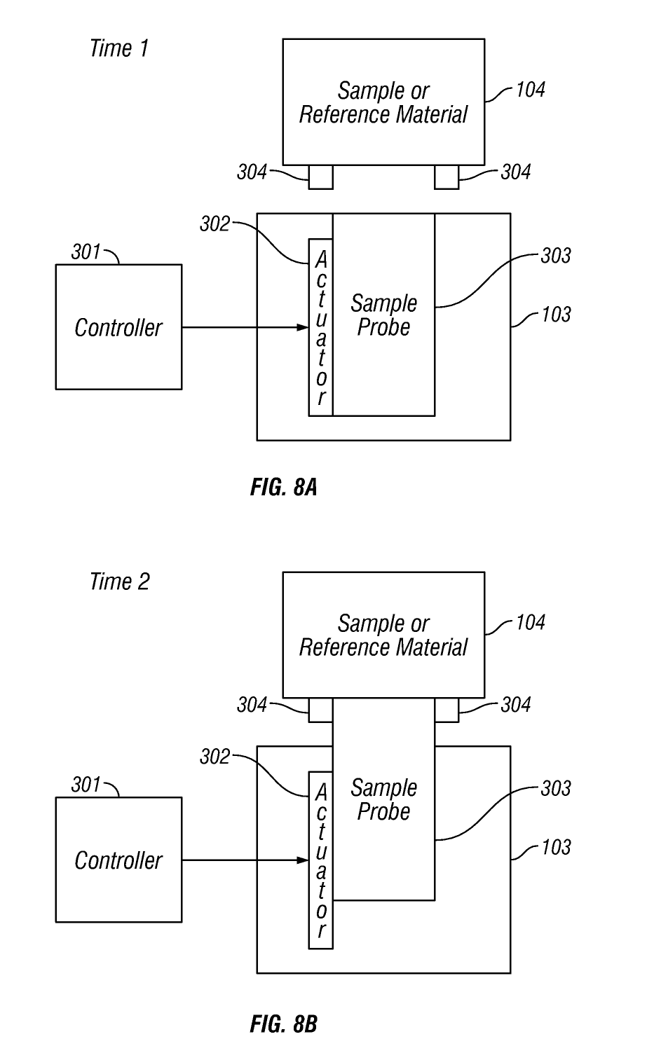

Referring now to FIGS. 8a and 8b, a schematic representation of sample probe control and sample probe movement relative to a sample is presented. The sample module 103 includes a sample probe 303. A controller 301 controls an actuator 302 that moves the sample probe 303. Signal processing means result in a control signal that is transferred from the controller 301 to the sample probe 303 typically through an actuator 302. The communicated control signal is used to control the z-axis movement of at least part of the sample module 103 relative to the tissue sample 104 or reference material. The part of the sample module 103 movable along at least the z-axis is referred to as the sample probe or sampling probe 303. In one case, the controller sends the control signal from the algorithm to the sample module actuator, preferably via a communication bundle. In a second case, the controller 301 receives input from the sample probe or other sensor and uses the input to move the actuator 302. Thus, in various embodiments, the controller is in different locations within the analyzer, such as in the sample module 103 or in the base module 101. In these cases, the actuator 302 subsequently moves the sample probe 303 relative to the tissue sample site 104. In a third case, no controller or actuator is used and the sample probe moves in response to an outside force, such as manual operation or due to gravity. The sample probe 303 is typically controlled along the z-axis from a position of no contact, to a position of proximate tissue sample contact, and optionally to a position of tissue sample displacement. The sample probe 303 is presented at a first (FIG. 8a) and second (FIG. 8b) instant of time with the first time presenting the sample probe when it is not in contact with the sample site. The second time presents the sample probe with minimal or nominal displacement of the sample tissue. The sample probe is, optionally, moved toward the sample, away from the sample, or remains static as a function of time as is discussed, infra. An optional guide 304 is attached to the sample and/or reference. Input to the controller 301 includes a predetermined profile, an interpretation of spectral data collected from the sample probe 303, or input from a sensor, such as a pressure sensor, an optical sensor, or a thermal sensor.

The intensity in both the second overtone spectral region, about 1100 to 1450 nm, and first overtone spectral region, about 1450 to 1900 nm, decreases in magnitude as the sample probe approaches and makes contact with the sample. Higher intensities represent non-contact of the sample probe with the sample. Intermediate intensities represent close proximity of a sample probe tip with the tissue sample or contact of the sample probe with a contact fluid, such as a fluorocarbon. Smaller intensities represent contact of the sample probe with the sample and/or displacement of the sample by the sampling probe.

Discrete intensity readings or intensity of spectra decrease as a sample probe moves toward contact with a skin sample site. After conversion to absorbance, it is observed that the absorbance increases as the sample probe moves toward the sample. This is largely the removal of specularly reflected light. For example, the light intensity approaches zero at 1450 nm where there is a large water absorbance band as the sample probe moves toward making contact with the sample. Generally, any high absorbance region, such as those due to water about 1450, 1900, and 2600 nm is usable to determine distance between a tip of the sample probe tip and the tissue sample as intensity decreases as the distance narrows and the corresponding absorbance increases as the distance between the sample probe tip and the tissue sample approaches zero. To enhance sensitivity to distance, a ratio or comparison of intensities at high intensity returning regions, such as those not dominated by water absorbance, and low intensity returning regions, such as those dominated by water, is used to estimate proximate or relative distance between the tip of the sample probe tip and the tissue sample.

X, Y, and Z-Axis Sample Probe Movement

Control of a noninvasive sample probe along the x, y, and z-axis is described in U.S. provisional patent application No. 60/658,708, which is incorporated herein in its entirety by this reference thereto.

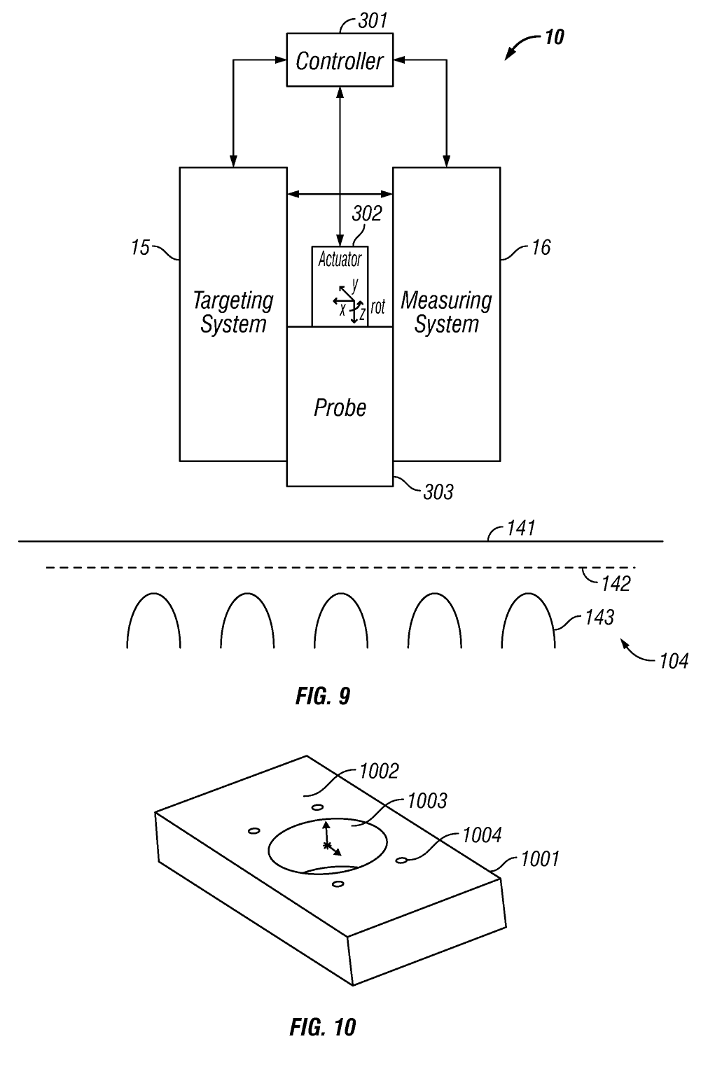

Referring now to FIG. 9, a block diagram of an analyzer having two primary systems, a targeting system 15 and a measuring system 16 is presented. The targeting system targets a tissue area or volume of the sample 104. For example, the targeting system targets a surface feature 141, one or more volumes or layers 142, and/or an underlying feature 143, such as a capillary or blood vessel. The measuring system contains a sample probe 303, which is optionally separate from or integrated into the targeting system. The sample probe of the measuring system is preferably directed to the targeted region or to a location relative to the targeted region either while the targeting system is active or subsequent to targeting. Less preferably, use of the measuring system is followed by use of the targeting system and a targeting image is used to post process the measuring system data. A controller 301 is used to direct the movement of the sample probe 303 in at least one of the x-, y-, and z-axes via one or more actuators 302. Optionally the controller directs a part of the analyzer that changes the observed tissue sample in terms of surface area or volume. The controller communicates with the targeting system, measuring system, and/or controller.

Sample Probe Tilt Control

Tilt orientation of the sample probe relative to the tissue sample is optionally controlled in conjunction with any of the above described x, y, and z-axes controls and/or with orientation or rotation control. For instance, the tilt of the sample probe relative to the x,y plane defined by the tissue sample is controlled. Several examples are presented here to illustrate tilt control of a noninvasive analyzer sample probe with optional x-, y-, or z-axis movement of the sample probe. The tilt or proximity control examples are illustrative and are not intended to limit the invention.

### EXAMPLE XXI

Piezoelectric devices are optionally used to move at least portion of a sample probe of a noninvasive analyzer relative to the tissue sample. For example a motor using a piezoelectric actuator is used as a drive. A piezoelectric actuator generates ultrasonic vibrations causing a threaded nut to vibrate in an orbit. The vibration results in rotation of a screw inside the nut thereby translating the rotary motion into a linear motion used as a drive. A piezoelectric motor has multiple advantages including: small size, quiet operation, smooth velocity, off-power hold, and programmable and/or feedback control. A piezoelectric motor is used to drive with about nanometer or about micrometer resolution. Generally, the piezoelectric motor is usable in place of electromagnetic stepper and servo gearhead motors, such as coils and solenoids. Thus, the piezoelectric motor is used to move the sample probe along the z-axis, translate about the x-axis and/or y-axis, to provide tilt of the sample probe by driving one edge of the sample probe at different relative speeds compared to other edges or sides of the sample probe, or convert linear travel of a lead screw to rotation. Piezoelectric devices are alternatively used to move a sample probe in terms of tilt and z-axis at the same time. Generally, piezoelectric drives are optionally used to control one or more of an x-axis, y-axis, z-axis, and tilt of an apparatus, such as all or part of a sample probe tip. Means of use of one or more piezoelectric motors include direct and indirect connection to a movable part or is connected in a fashion adapting linear to rotational movement. An exemplary piezoelectric system is a hexapod, such as the M-850 hexapod 6-axis parallel kinematics robot (Physik, Instrumente, Tustin, Calif.)

### EXAMPLE XXII

Referring now to FIG. 10, an embodiment is presented having a proximity sensor with a plurality of detection points for estimating distance to contact with a sample site and/or contact with the sample site. In this example, a sample probe tip 1001 is illustrated. One side of the sample probe tip 1002 is brought in proximate contact with a sample tissue site. A collection fiber is inserted into the center of the probe tip for noninvasive sample photon collection with incident photons hitting the tissue sample between the central collection fiber and the outer opening of the central opening 1003. The four holes 1004 surrounding the central opening are for proximity sensing. In one case, incident photons from the central opening 1003 are detected by detectors located in the four openings 1004 or are directed to detectors via light redirection optics, such as fiber optics, to one or more detectors optically associated with the four openings 1004. Detected spectra, discrete wavelengths, averaged intensity across wavelengths, or discrete intensities or metrics derived therefrom are then used to determine proximity to the tissue sample and/or contact with the tissue sample as described, supra, for one or more of the collected signals. Having multiple collection areas allows the relative tilt of the sample probe relative to the tissue sample to be determined. For example, spectrally determined contact at a first collection site while specular reflectance is still dominating at a second collection site is indicative of the sample probe tilting toward the first collection site. Feedback control of this indication allows the actuators to lift the first side of the sample probe associated with the first sample site and/or the actuators to lower the side of the sample probe associated with the second collection site. Thus the sample probe is leveled or made orthogonal relative to the tissue sample. Having multiple detection sites allows tilt of the sample probe to be adjusted relative to the tissue sample along a plane, such as an x,y plane as opposed to along a line. Preferably, multiple detection sites are used to dynamically adjust tilt of the sample probe. For example, three detection sites allow tilt control relative to a plane. Preferably, four detection sites are used to control sample probe tilt. Preferably, two detection sites lie along the x-axis, such as along a forearm. This allows the sample probe to be aligned relative to the length of the sample site compensating for the radius of curvature about the y-axis of the tissue sample. Preferably, two additional detectors along the y-axis of the sample probe adjust tilt or control proximity of the sample probe along the y-axis. Notably, not all of the plurality of sensors are forced to have the same intensity reading for proximity sensing. For example, contact with detectors along the x-axis and no contact along the y-axis is indicative of the probe making contact with the sample in one dimension. Balancing the returned intensities along the y-axis controls tilt relative to the curved sample tissue. Additionally, the noninvasive probe collected light for analyte property determination is used with one or more radially aligned detectors to adjust tilt of the sample probe relative to the tissue. Additional algorithms for proximity sensing are disclosed in U.S. patent application Ser. No. 11/117,104, and U.S. provisional patent application No. 60/658,708, which are both incorporated herein in their entirety by this reference thereto.

In an additional embodiment, the relative distance of different edges of the sample probe tip to the tissue sample is provided via one or more capacitance readings. For example, contact of one or more regions of a sample probe tip with the sample is provided via touch screen technology, which is technology based on charge-transfer methods. A matrix scanning touch screen system uses a pulse driven vector, such as a row, and charge receiving vector, such as a column, of traces. When a key or region is touched, some of the charge is diverted and absorbed by the human body. As a result, the amount of charge pumped into the receiving electrode drops. The drop in signal is interpreted as touch. In the current invention, one or more touch sensors are placed onto the end of a sample probe to allow detection of contact with a tissue sample site. Preferably, a plurality of touch sensors are distributed about the surface of the sample probe. In this manner, based upon detected touch, the tilt of the sample probe is determined. The sample probe is either backed off and adjusted based upon the detected touch area or the opposite side of the sample probe is preferentially driven down the z-axis to touch or come into close proximity with the sample. In the preferred embodiment, four capacitive sensors are distributed evenly around the center of the probe tip to detect distance from the arm. Readings from different sensors are used to adjust the probe angle until the desired angle is reached. In this embodiment, the capacitance is altered prior to the tissue actually touching the screen allowing the sample probe to be adjusted in terms of at least tilt prior to initial contact with the sample.

Two mechanical embodiments allowing tilt control are provided herein that are illustrative of controlling sample probe x, y, and/or z-axis positioning and/or tilt relative to a tissue sample. The mechanical embodiments are illustrative in nature and are not intended to limit the scope of possible mechanical means of sample probe mechanical movement control. Drivers for sample probe movement include traditional drivers and the piezoelectric based actuators described, supra. Notably, the drivers move the entire sample probe, a portion of the sample probe, or control tilt by moving one side of the sample probe to a different vertical position off of the sample site relative to one or more additional sides of the sample probe.

Gimbal Tilt Orientation

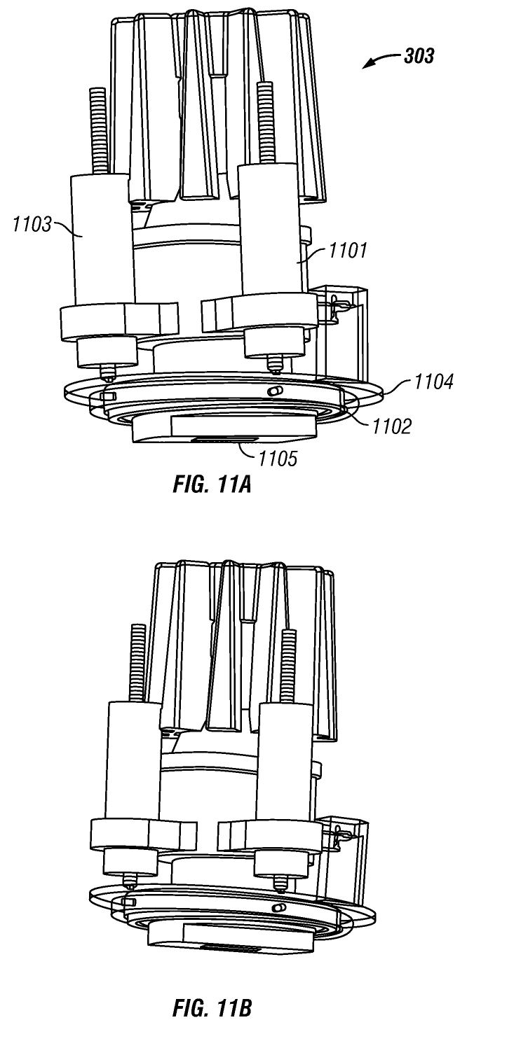

Referring now to FIG. 11A and FIG. 11B, an embodiment having a sample probe 303 that is tilt adjustable using a gimbal ring is presented. The sample probe 303 has a sample probe tip 1105 that interfaces with a tissue sample site. The gimbal includes a first ring 1102 and a second ring 1104. The rings tilt on separate axes. A first drive 1101 pushes on the first ring 1102 causing the sample probe 303 to tilt about a first axis, such as the x-axis. A second drive 1103 pushes on the second ring 1104 causing the sample probe 303 to tilt about a second axis, such as the y-axis. Combined, the system allows tilt control of the sample probe relative to the z-axis. FIG. 11A illustrates the sample probe in a first tilt state and FIG. 11B illustrates the sample probe in a second tilt state.

Spherical Tilt

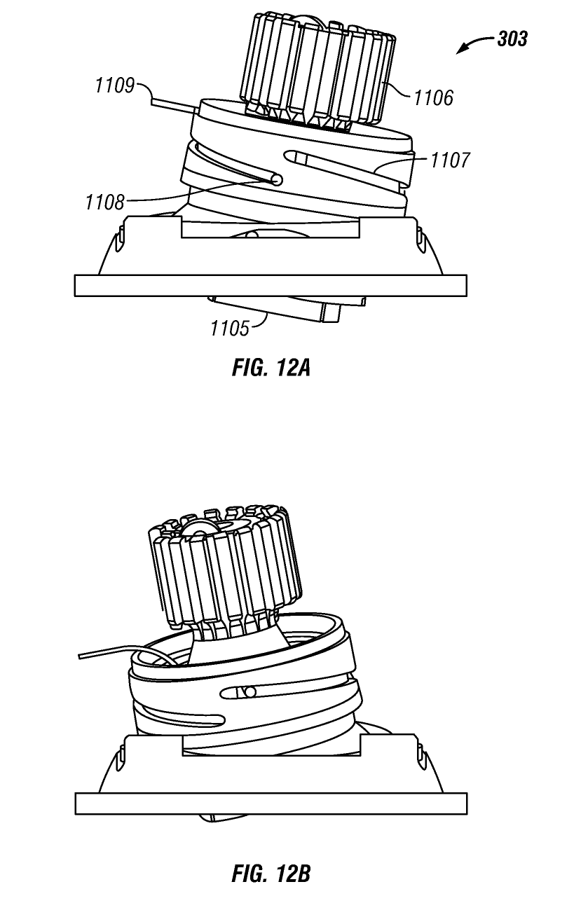

Referring now to FIG. 12A and FIG. 12B, an embodiment is presented having a sample probe 303 in two tilt configurations, respectively. The sample probe 303 includes: an internal source; a sample probe tip 1105 for interfacing with a sample, such as a tissue sample; a collection fiber 1109; a slot containing element 1107; and a pin 1108. As the slot housing element is rotated the pin 1108 moves the slot containing element which in turn moves the sample probe. In FIG. 12A, the sample probe tip 1105 is extended from the end of the sample probe 303. In FIG. 12B, the sample probe tip 1105 is retracted toward the end of the sample probe 303. Thus the slot containing element and pin translate at least a portion of the sample probe 303 along the z-axis. Optionally, translation is performed at the same time the sample probe tip is tilt controlled. In this example, the tilt of the sample probe tilts along a sphere relative to a central sphere point, preferably at the center of the probe tip 303 or down the z-axis from the center of the probe tip. In FIG. 12A, the sample probe tip 1105 is shown in a first tilt state. In FIG. 12B, the sample probe tip 1105 is shown in a second tilt state. This configuration illustrates tilt control at the same time as z-axis control of the sample probe tip relative to a sample site.

Permutations, combinations, and obvious variants of the above described invention are also included in this invention. For example, apparatus and methodologies taught for a given analyte are applicable to additional noninvasive analytes. Permutations and combinations of methods and apparatus for photonic-stimulation sources described in this section are used in conjunction with analyzers or incorporated into analyzers. Further, sample probe movements are optionally combined together and are useable with any of the photostimulation techniques described herein.

Those skilled in the art will recognize that the present invention may be manifested in a variety of forms other than the specific embodiments described and contemplated herein. Departures in form and detail may be made without departing from the spirit and scope of the present invention. Accordingly, the invention should only be limited by the Claims included below.

1. A method for analyte property determination at a tissue sample site of a human subject, comprising the steps of:
generating a calibration from samples collected from well perfused tissue;
enhancing perfusion at the sample site by photostimulation at or near the sample site with a first photon source;
noninvasively measuring a spectrum from the sample site, wherein said step of measuring uses a second photon source; and
estimating said analyte property using said calibration and said spectrum, wherein said calibration generated using samples collected from well perfused sample tissue applies to said spectrum.

2. The method of claim 1, wherein said well perfused tissue comprises tissue from a plurality of calibration subjects, wherein the sample site comprises tissue from a measurement subject.

3. The method of claim 2, wherein said measurement subject is not a member of said plurality of calibration subjects.

4. The method of claim 2, wherein said well perfused tissue comprises tissue from a fingertip or a toe, wherein the sample site comprises a skin/tissue sample that does not comprise a region of the measurement subject's fingertip or toe.

5. The method of claim 1, wherein said well perfused tissue comprises any of:
photostimulated tissue;
finger tissue; and
toe tissue.

6. The method of claim 5, wherein the sample site does not comprise a fingertip or a toe.

7. The method of claim 1, wherein the sample site comprises a tissue volume having at least intermittent degradation of tissue perfusion compared to perfusion of a fingertip.

8. The method of claim 1, wherein said step of using a first photon source occurs prior to said measuring step.

9. The method of claim 8, wherein said step of using a first photon source is repeated over a period of at least days to produce angiogenesis at or about the sample site before said step of noninvasively measuring.

10. The method of claim 1, wherein said spectrum represents photons from said second source in the absence of photons from said first source.

11. The method of claim 1, wherein said first source comprises a light emitting diode.

12. The method of claim 11, wherein said second source comprises a broadband source.

13. The method of claim 1, wherein said calibration comprises a multivariate model.

14. The method of claim 13, wherein said multivariate model uses at least one reading from each of at least ten wavelengths.

15. The method of claim 1, further comprising a step of using a second perfusion enhancement technique, wherein said photostimulation step occurs within four hours prior to said noninvasively measuring step, wherein said second perfusion enhancement technique is used after said photostimulation step, and wherein said second perfusion enhancement technique comprises any of:
applying additional heat to the sample site beyond that of photostimulation to the sample site;
rubbing at or about the sample site;
ingestion, by the subject, of L-arginine;
ingestion, by the subject, of a surface capillary dilating agent;
applying a negative pressure at or about the sample site; and
application of a topical vasodilating agent at the sample site.

16. The method of claim 1, wherein said well perfused tissue comprises tissue that is intermittently not well perfused, wherein said tissue that is not well perfused is subjected to photostimulation prior to generation of said calibration to enhance perfusion.

17. A method for analyte property determination at a sample site of a human subject, comprising the steps of:
enhancing perfusion at the sample site by photostimulating about the sample site;
enhancing perfusion of the sample site with a second technique, wherein said second technique is used within four hours of said photostimulating step; and
determining said analyte property with either an invasive apparatus or a noninvasive apparatus after said steps of photostimulating.

18. The method of claim 17, wherein said second technique comprises any of:
applying additional heat beyond that of photostimulation to the sample site;
rubbing at or about the same site;

19. The method of claim 17, wherein said second technique comprises intake of L-arginine by the subject.

20. The method of claim 17, wherein said second technique comprises any of:
intake of a surface capillary dilating agent by the subject;
applying a negative pressure at or about the sample site; and
application of a topical pharmacologic or vasodilating agents to the sample site.

21. The method of claim 17, further comprising the step of:
determining a glucose concentration of the subject in a biological sample collected from a body part of the subject comprising any of:
a forearm;
an upper arm;
a head;
a torso;
an abdominal region;
a thigh; and
a calf.

22. The method of claim 21, wherein said invasive apparatus comprises an alternative invasive apparatus, wherein said alternative invasive apparatus acquires a biological sample from the subject using any of:
laser poration;
applied current; and
a partial vacuum.

23. A method for analyte property determination at a sample site, of a human subject comprising the steps of:
enhancing perfusion at the sample site by photostimulating a region about the sample site;
noninvasively determining said analyte property at the sample site, wherein said determining step is performed within a period of four hours following said photostimulating step, wherein said noninvasively determining step uses at least one wavelength of incident light not used in said photostimulating step.

24. A method for analyte property determination at a sample site of a human subject, comprising the steps of:
enhancing perfusion at the sample site by photostimulating with a light emitting diode at or about the sample site;
noninvasively determining said analyte property at said photostimulated sample site, wherein said determining step is performed using a photon source having a total spectral range in excess of 300 nm, wherein said determining step occurs within four hours of said photostimulating step.

25. An apparatus for analyte property determination at a tissue sample site of a human subject comprising:
a first photon source for photostimulation at or near the sample site to enhance perfusion at the sample site; and
an analyzer comprising a calibration generated using samples collected from well perfused tissue, said analyzer comprising a second photon source, means for measuring a spectrum from the sample site, and means for estimating said analyte property using said calibration and said spectrum,
wherein samples collected from well perfused sample tissue are used to generate said calibration that is applied to said spectrum.

26. The apparatus of claim 25, wherein said first photon source comprises at least one light emitting diode, and said second photon source comprises a broadband source providing light over a wavelength range of at least 300 nm.

27. The apparatus of claim 25, wherein said analyzer further comprises:
a sample probe tip; and
means for z-axis control of said sample probe tip relative to the sample site.

28. The apparatus of claim 26, wherein said analyzer further comprises:
means for tilt control of at least a portion of said analyzer relative to the sample site.

29. The apparatus of claim 25, wherein said analyzer further comprises a multivariate model, wherein said multivariate model receives as an input at least one reading from each of at least ten wavelengths.

30. The apparatus of claim 25, wherein said analyzer further comprises a second means for enhancing perfusion at the sample site.

31. The apparatus of claim 25, wherein said first photon source for photostimulation is integrated into said analyzer.
